SEQUENCE LISTING (3 sequences)

SEQ ID NO: 1 - protein, 21 aa, Artificial Sequence
  A Chain of the human insulin analog
GIVEQCCTSICSLEQLENYCN

SEQ ID NO: 2 - protein, 29 aa, Artificial
  B Chain of the human insulin analog
FVNQHLCGSHLVEALELVCGERGFHYTPK

SEQ ID NO: 3 - protein, 29 aa, Artificial
  B Chain of the human insulin analog
FVNQHLCGSHLVEALHLVCGERGFHYTPK

(19) United States
(12) Patent Application Publication
(10) Pub. No.: US 2024/0374692 A1
(43) Pub. Date: Nov. 14, 2024

(54) A NOVEL ACYLATED INSULIN ANALOG

(71) Applicant: SUNSHINE LAKE PHARMA CO., LTD., Dongguan, Guangdong (CN)

(72) Inventors: Baoye ZHENG, Dongguan (CN); Dan LEI, Dongguan (CN); Haigang WANG, Dongguan (CN); Shushan LIN, Dongguan (CN); Zhizhu ZHAN, Dongguan (CN); Qian WANG, Dongguan (CN); Qiuyan LIU, Dongguan (CN); Yangling HU, Dongguan (CN); Zilan YANG, Dongguan (CN); Yan JIANG, Dongguan (CN); Wenjia LI, Dongguan (CN)

(73) Assignee: SUNSHINE LAKE PHARMA CO., LTD., Dongguan, Guangdong (CN)

(21) Appl. No.: 18/563,090

(22) PCT Filed: May 23, 2022
(86) PCT No.: PCT/CN2022/094392

§ 371 Date: Nov. 21, 2023

(30) Foreign Application Priority Data
May 24, 2021 (CN) 202110570030.6

(51) Int. Cl. A61K 38/28; A61K 47/54; A61P 3/10; C07D 207/46; C07K 1/107

(52) U.S. Cl. CPC A61K 38/28; A61K 47/542; A61K 47/545; A61P 3/10; C07D 207/46; C07K 1/1077

(57) ABSTRACT

A novel acylated insulin analog is provided as well as a side chain compound that can be used to prepare the acylated insulin analog, a pharmaceutical composition thereof, a pharmaceutical use, an administration method and a preparation method. The acylated insulin analog can be used for the treatment of diabetes. It has the effect as a weekly preparation or a longer-acting insulin preparation and can be used for the treatment once a week or less frequently, increasing the compliance of diabetic patients.

### CROSS-REFERENCE TO RELATED APPLICATION

This application claims the priority and benefits of Chinese Patent Application No. 202110570030.6, filed with the State Intellectual Property Office of China on May 24, 2021, which is incorporated herein by reference in its entirety.

### TECHNICAL FIELD

The present invention relates to the field of biopharmaceuticals. In particular, it relates to a novel acylated insulin analog. More particularly, it relates to a side chain compound that can be used to prepare an acylated insulin analog, an acylated insulin analog, and pharmaceutical compositions, pharmaceutical uses, administration methods and preparation methods thereof.

### BACKGROUND ART

The treatment of diabetes, both type I and type II, is increasingly reliant on so-called potent insulin therapy. Under this regimen, patients are treated with multiple daily insulin injections, including using long-acting insulin injections once or twice a day to cover basal insulin needs, and supplemented with large amount of fast-acting insulin to cover meal-related insulin need.

Many diabetic patients need insulin injection 2-4 times per day, and weekly, monthly and yearly like this. Patient compliance is poor, and long-term subcutaneous injections cause some damage to the skin, the discomfort of large daily injections can be reduced by using longer-acting insulin analogs, thus there is a need for an insulin analog that can be injected at least once a week.

CN105636979 discloses a new derivative of insulin analogs, but its action time is still not ideal, a basal insulin preparation administered once a week or even less frequently is still urgently needed.

### SUMMARY OF THE INVENTION

The object of the present invention is to overcome or ameliorate at least one disadvantage of the prior art, or to provide a useful alternative.

In the first aspect of the present invention, provided herein is a novel side chain compound having the structure shown in formula (I):

W—X—Y—Z—R (I)

W is a fatty acid or fatty diacid with 10-20 carbon atoms, the structure is —CO(CH₂)ₙCOOH, and n is an integer between 10-20;
X is a diamino compound containing a carboxylic acid group, wherein the carbon atom connecting the carboxylic acid group can be a chiral carbon or an achiral carbon, and has the structures shown in formulas (a1), (a2) and (a3),

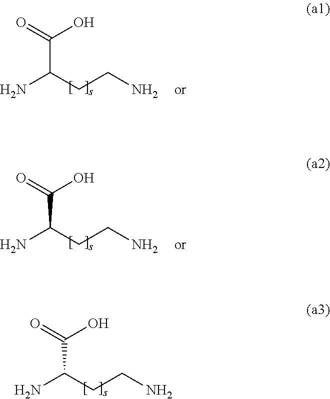

wherein s is an integer between 2-20; in some embodiments, s is 2-10; in other embodiments, s is 2-8; in still other embodiments, s is 4; one of the amino groups in X is connected with one of the acyl groups in W to form an amide bond;
Y is -A(CH₂)ₘB—, wherein m is an integer between 1-10; in some embodiments, m is an integer between 1-6; in some embodiments, m is 2; A and B are absent or are —CO—.

Z is —(OEG)ₚ, p is an integer between 1-3; in some embodiments, p is 2, and the OEG structure is

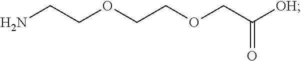 [SMILES: NCCOCCOCC(=O)O]

or, n can also be an integer between 4-30. R is a leaving group; in some embodiments, R is an activated ester group;
the linking groups between W, X, Y and Z are amide peptide bonds or peptide bonds.

Further, the side chain compound of the present invention may have the following structural formulas:

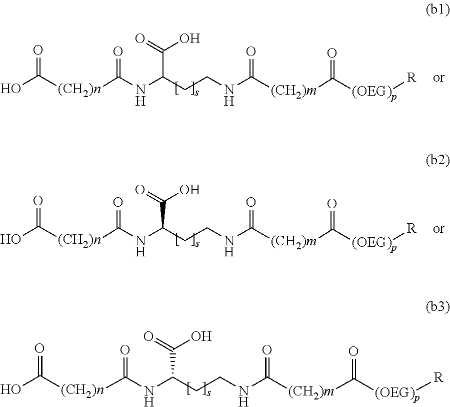

wherein, n is an integer between 14-20, s is an integer between 2-4, m is an integer between 1-4, p is 2,
R is selected from the following groups:

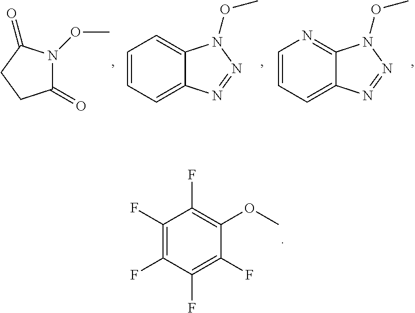

In some embodiments of the present invention, n is an integer between 14-20, s is an integer between 2-4, m is 2, p is 2,
R is:

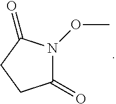

In some embodiments of the present invention, the side chain compound has the following structural formulas:

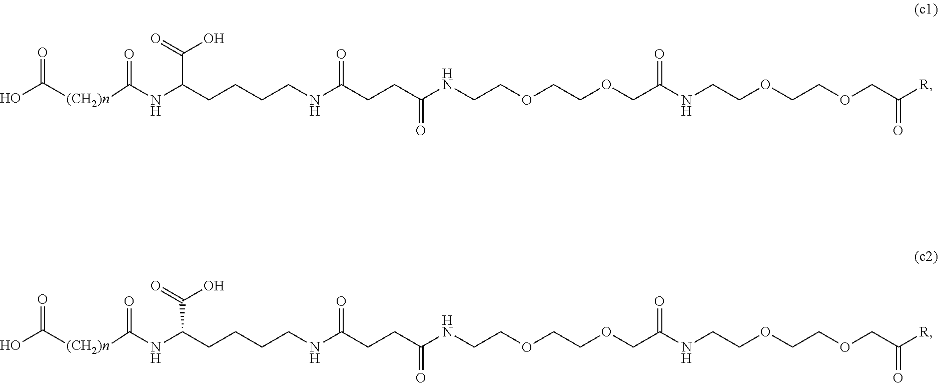

wherein, n is an integer between 16-18, R is:

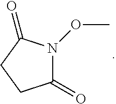

In some embodiments of the present invention, the side chain compound of the present invention is selected from any one of the following compounds:

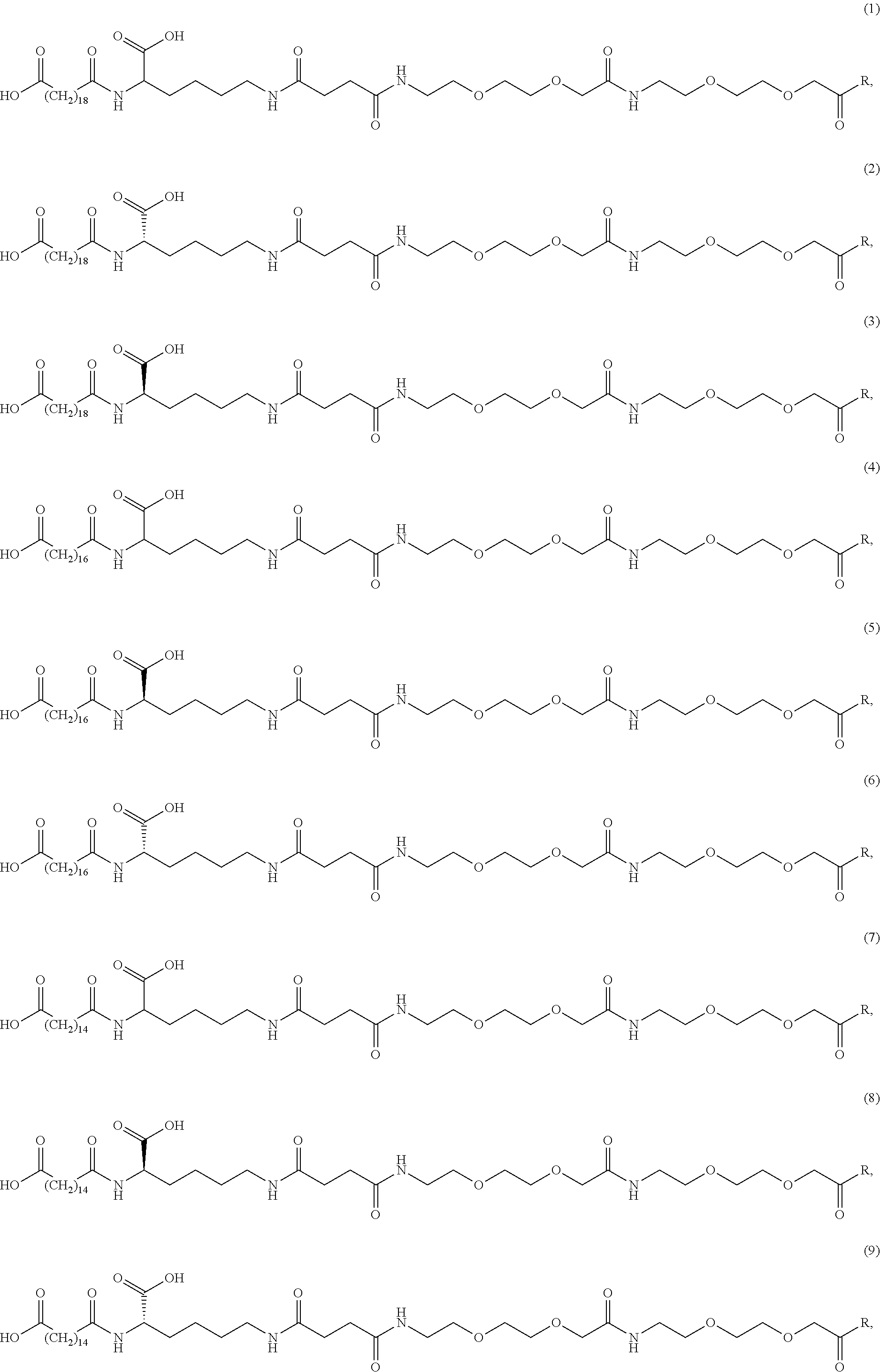

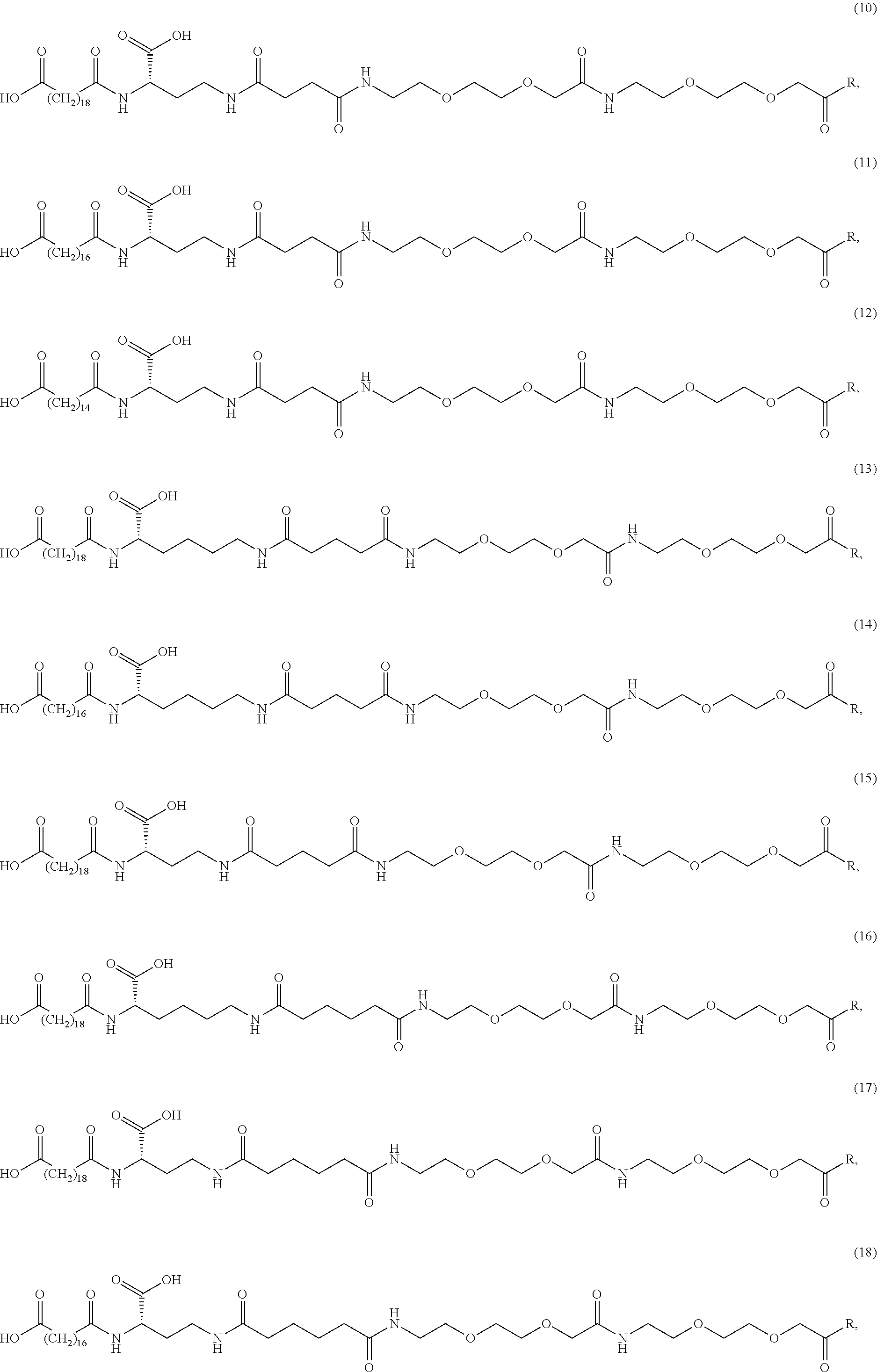

wherein, R is

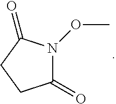

In still other embodiments of the present invention, the side chain compound has the following structural formulas:

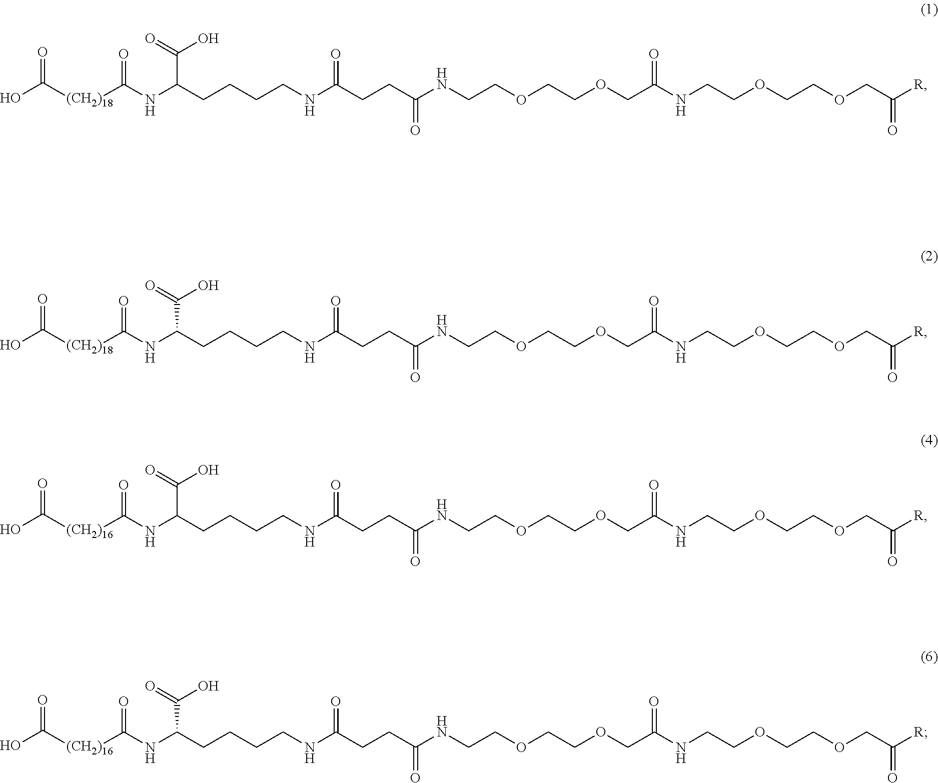

wherein, R is

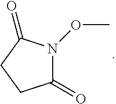

In still other embodiments of the present invention, the side chain compound has the following structural formulas:

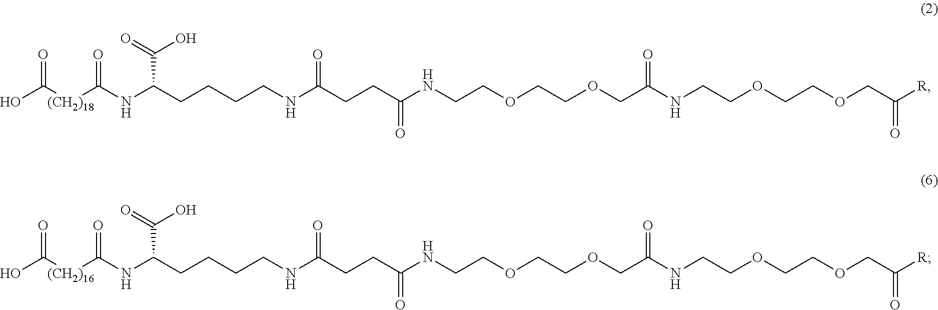

wherein, R is

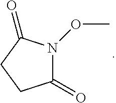

In the second aspect of the present invention, a novel acylated insulin analog is proposed, which is obtained by an acylation reaction between the side chain compound of the present invention and a human insulin analog, and the structure is shown in formula (II):

W—X—Y—Z-M (II)
wherein:
W is a fatty acid or fatty diacid with 10-20 carbon atoms, the structure is —CO(CH₂)ₙCOOH, and n is an integer between 10-20;
X is a diamino compound containing a carboxylic acid group, wherein the carbon atom connecting the carboxylic acid group can be a chiral carbon or an achiral carbon, and has the structures shown in formulas (a1), (a2) and (a3),

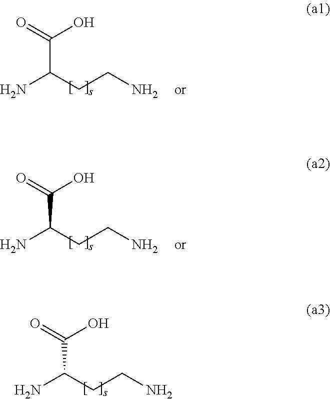

wherein s is an integer between 2-20, in some embodiments, s is 2-10, in other embodiments, s is 2-8, one of the amino groups in X is connected with one of the acyl groups in W to form an amide bond;
Y is -A(CH₂)ₘB—, wherein m is an integer between 1-10, in some embodiments, m is an integer between 1-6, A and B are absent or are —CO—;
Z is —(OEG)ₚ, p is an integer between 1-3, in some embodiments, p is 2, and the OEG structure is

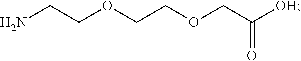 [SMILES: NCCOCCOCC(=O)O]

in other embodiments, p can be an integer between 4-30.

The linking groups between W, X, Y and Z are amide (peptide) bonds;
M is a human insulin analog.

In some embodiments of the present invention, the acylated insulin analog has a side chain compound of the following structures:

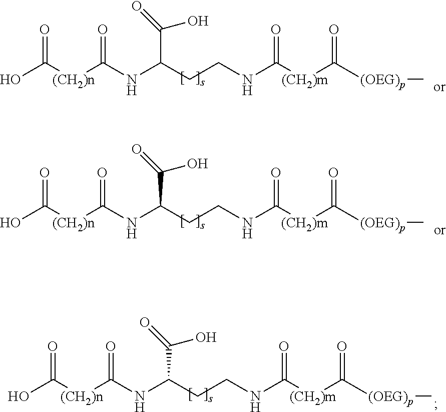

wherein, n is an integer between 14-20, s is an integer between 2-8, m is an integer between 1-6, and p is an integer between 1-3.

In some embodiments of the present invention, the acylated insulin analog has a side chain compound of the following structures:

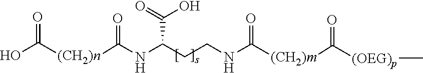

wherein, n is an integer between 14-20, s is an integer between 2-8, m is an integer between 1-6, and p is an integer between 1-3.

The acylated insulin analog of the present invention is obtained by an acylation reaction between the side chain compound of the present invention and a human insulin analog, wherein the human insulin analog has A chain and B chain, the amino acid sequence of the A chain is shown in SEQ ID NO.1, the amino acid sequence of the B chain is shown in SEQ ID NO.2 or SEQ ID NO.3, and the human insulin analog is connected to the side chain compound by an amide bond through the ε nitrogen of the lysine residue at position B29.

A Chain:
(SEQ ID NO. 1)
GIVEQCCTSICSLEQLENYCN
B Chain:
(SEQ ID NO. 2)
FVNQHLCGSHLVEALELVCGERGFHYTPK
B Chain:
(SEQ ID NO. 3)
FVNQHLCGSHLVEALHLVCGERGFHYTPK

In some embodiments of the present invention, the acylated insulin analog of the present invention have the following structural formulas:
A14E, B16E, B25H, B29K(N(ε)-COOH(CH₂)ₙCO—NHC(COOH)(CH₂)SCH₂NH—CO(CH₂)ₘCO—(OEG)ₚ), desB30 human insulin analog, or,
A14E, B16H, B25H, B29K(N(ε)-COOH(CH₂)ₙCO—NHC(COOH)(CH₂)SCH₂NH—CO(CH₂)ₘCO—(OEG)ₚ), desB30 human insulin analog;
wherein, n is an integer between 14-20, s is an integer between 2-8, m is an integer between 1-6, and p is 2; it should be noted that the C atom connecting the carboxyl group in —NHC(COOH)(CH₂)SCH₂NH— can be in D form, L form or racemic form.

In some embodiments of the present invention, n is an integer between 14-18, s is an integer between 3-4, m is an integer between 2-4, and p is 2.

Further, the acylated insulin analog of the present invention is selected from any one of the following compounds:
A14E, B16E, 25H, B29K(N(ε)-COOH(CH₂)₁₈CO-Lys-CO(CH₂)₂CO—(OEG)₂), desB30 human insulin analog,
A14E, B16E, B25H, B29K(N(ε)-COOH(CH₂)₁₈CO-L-Lys-CO(CH₂)₂CO—(OEG)₂), desB30 human insulin analog,
A14E, B16E, B25H, B29K(N(ε)-COOH(CH₂)₁₈CO-D-Lys-CO(CH₂)₂CO—(OEG)₂), desB30 human insulin analog,
A14E, B16E, B25H, B29K(N(ε)-COOH(CH₂)₁₆CO-Lys-CO(CH₂)₂CO—(OEG)₂), desB30 human insulin analog,
A14E, B16E, B25H, B29K(N(ε)-COOH(CH₂)₁₆CO-D-Lys-CO(CH₂)₂CO—(OEG)₂), desB30 human insulin analog,
A14E, B16E, B25H, B29K(N(ε)-COOH(CH₂)₁₆CO-L-Lys-CO(CH₂)₂CO—(OEG)₂), desB30 human insulin analog,
A14E, B16E, B25H, B29K(N(ε)-COOH(CH₂)₁₄CO-Lys-CO(CH₂)₂CO—(OEG)₂), desB30 human insulin analog,
A14E, B16E, B25H, B29K(N(ε)-COOH(CH₂)₁₄CO-D-Lys-CO(CH₂)₂CO—(OEG)₂), desB30 human insulin analog,
A14E, B16E, B25H, B29K(N(ε)-COOH(CH₂)₁₄CO-L-Lys-CO(CH₂)₂CO—(OEG)₂), desB30 human insulin analog,
A14E, B16E, B25H, B29K(N(ε)-COOH(CH₂)₁₈CO-L-Dab-CO(CH₂)₂CO—(OEG)₂), desB30 human insulin analog,
A14E, B16E, B25H, B29K(N(ε)-COOH(CH₂)₁₆CO-L-Dab-CO(CH₂)₂CO—(OEG)₂), desB30 human insulin analog,
A14E, B16E, B25H, B29K(N(ε)-COOH(CH₂)₁₄CO-L-Dab-CO(CH₂)₂CO—(OEG)₂), desB30 human insulin analog,
A14E, B16E, B25H, B29K(N(ε)-COOH(CH₂)₁₈CO-L-Lys-CO (CH₂)₃CO—(OEG)₂), desB30 human insulin analog,
A14E, B16E, B25H, B29K(N(ε)-COOH(CH₂)₁₆CO-L-Lys-CO (CH₂)₃CO—(OEG)₂), desB30 human insulin analog,
A14E, B16E, B25H, B29K(N(ε)-COOH(CH₂)₁₈CO-L-Dab-CO(CH₂)₃CO—(OEG)₂), desB30 human insulin analog,
A14E, B16E, B25H, B29K(N(ε)-COOH(CH₂)₁₈CO-L-Lys-CO (CH₂)₄CO—(OEG)₂), desB30 human insulin analog,
A14E, B16E, B25H, B29K(N(ε)-COOH(CH₂)₁₈CO-L-Dab-CO(CH₂)₄CO—(OEG)₂), desB30 human insulin analog,
A14E, B16E, B25H, B29K(N(ε)-COOH(CH₂)₁₆CO-L-Dab-CO(CH₂)₄CO—(OEG)₂), desB30 human insulin analog,
A14E, B16H, B25H, B29K(N(ε)-COOH(CH₂)₁₈CO-Lys-CO(CH₂)₂CO—(OEG)₂), desB30 human insulin analog,
A14E, B16H, B25H, B29K(N(ε)-COOH(CH₂)₁₈CO-L-Lys-CO(CH₂)₂CO—(OEG)₂), desB30 human insulin analog,
A14E, B16H, B25H, B29K(N(ε)-COOH(CH₂)₁₈CO-D-Lys-CO(CH₂)₂CO—(OEG)₂), desB30 human insulin analog,
A14E, B16H, B25H, B29K(N(ε)-COOH(CH₂)₁₆CO-Lys-CO(CH₂)₂CO—(OEG)₂), desB30 human insulin analog,
A14E, B16H, B25H, B29K(N(ε)-COOH(CH₂)₁₆CO-D-Lys-CO(CH₂)₂CO—(OEG)₂), desB30 human insulin analog,
A14E, B16H, B25H, B29K(N(ε)-COOH(CH₂)₁₆CO-L-Lys-CO(CH₂)₂CO—(OEG)₂), desB30 human insulin analog,
A14E, B16H, B25H, B29K(N(ε)-COOH(CH₂)₁₄CO-Lys-CO(CH₂)₂CO—(OEG)₂), desB30 human insulin analog,
A14E, B16H, B25H, B29K(N(ε)-COOH(CH₂)₁₄CO-D-Lys-CO(CH₂)₂CO—(OEG)₂), desB30 human insulin analog,
A14E, B16H, B25H, B29K(N(ε)-COOH(CH₂)₁₄CO-L-Lys-CO(CH₂)₂CO—(OEG)₂), desB30 human insulin analog,
A14E, B16H, B25H, B29K(N(ε)-COOH(CH₂)₁₈CO-L-Dab-CO(CH₂)₂CO—(OEG)₂), desB30 human insulin analog,
A14E, B16H, B25H, B29K(N(ε)-COOH(CH₂)₁₆CO-L-Dab-CO(CH₂)₂CO—(OEG)₂), desB30 human insulin analog,
A14E, B16E, B25H, B29K(N(ε)-COOH(CH₂)₁₄CO-L-Dab-CO(CH₂)₂CO—(OEG)₂), desB30 human insulin analog,
A14E, B16H, B25H, B29K(N(ε)-COOH(CH₂)₁₈CO-L-Lys-CO (CH₂)₃CO—(OEG)₂), desB30 human insulin analog,
A14E, B16H, B25H, B29K(N(ε)-COOH(CH₂)₁₆CO-L-Lys-CO (CH₂)₃CO—(OEG)₂), desB30 human insulin analog,
A14E, B16H, B25H, B29K(N(ε)-COOH(CH₂)₁₈CO-L-Dab-CO(CH₂)₃CO—(OEG)₂), desB30 human insulin analog,
A14E, B16H, B25H, B29K(N(ε)-COOH(CH₂)₁₈CO-L-Lys-CO (CH₂)₄CO—(OEG)₂), desB30 human insulin analog,
A14E, B16H, B25H, B29K(N(ε)-COOH(CH₂)₁₈CO-L-Dab-CO(CH₂)₄CO—(OEG)₂), desB30 human insulin analog,
A14E, B16H, B25H, B29K(N(ε)-COOH(CH₂)₁₈CO-L-Dab-CO(CH₂)₄CO—(OEG)₂), desB30 human insulin analog;
“-Lys-” means connection via achiral lysine, “-L-Lys-” means connection via L chiral lysine, “-D-Lys-” means connection via D chiral lysine;
“Dab” means 2,4-diaminobutyric acid. “-L-Dab-” means connection via L chiral Dab, and “-D-Dab-” means connection via D chiral lysine.

In some embodiments of the present invention, the acylated insulin analog is selected from any one of the following compounds:
A14E, B16E, B25H, B29K(N(ε)-COOH(CH₂)₁₈CO-Lys-CO(CH₂)₂CO—(OEG)₂), desB30 human insulin analog,
A14E, B16E, B25H, B29K(N(ε)-COOH(CH₂)₁₈CO-L-Lys-CO(CH₂)₂CO—(OEG)₂), desB30 human insulin analog,
A14E, B16H, B25H, B29K(N(ε)-COOH(CH₂)₁₈CO-Lys-CO (CH₂)₂CO—(OEG)₂), desB30 human insulin analog; A14E, B16H, B25H, B29K(N(ε)-COOH(CH₂)₁₈CO-L-Lys-CO (CH₂)₂CO—(OEG)₂), desB30 human insulin analog.

In yet other embodiments of the present invention, the acylated insulin analog can be selected from any one of the following compounds:
A14E, B16E, B25H, B29K(N(ε)-COOH(CH₂)₁₈CO-L-Lys-CO(CH₂)₂CO—(OEG)₂), desB30 human insulin analog,
A14E, B16H, B25H, B29K(N(ε)-COOH(CH₂)₁₈CO-L-Lys-CO (CH₂)₂CO—(OEG)₂), desB30 human insulin analog.

Wherein, A14E, B16E, B25H, B29K(N(ε)-COOH(CH₂)₁₈CO-L-Lys-CO(CH₂)₂C O—(OEG)₂), desB30 human insulin analog has the structure shown in the following formul a:

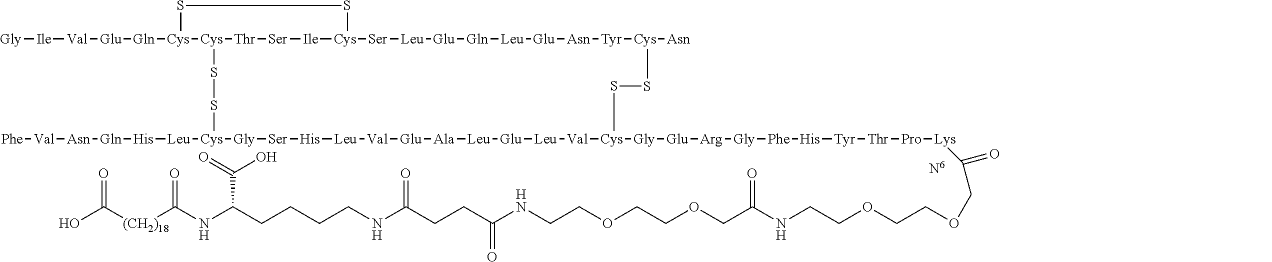

A14E, B16H, B25H, B29K(N(ε)-COOH(CH₂)₁₈CO-L-Lys-CO (CH₂)₂CO—(OEG)₂), desB30 human insulin analog has the structure shown in the following formula:

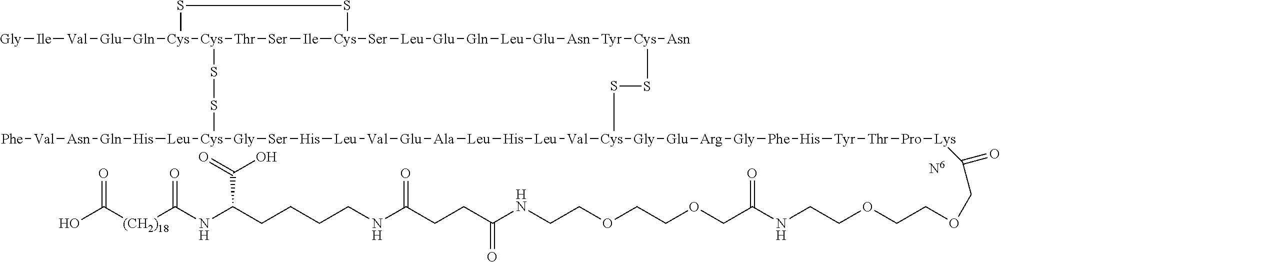

The third aspect of the present invention proposes a pharmaceutical composition comprising the side chain compound and the acylated insulin analog of the present invention.

The fourth aspect of the present invention proposes use of the side chain compound, acylated insulin analog and pharmaceutical composition of the present invention in the manufacture of a medicament for treating or preventing diabetes in a subject; the diabetes refers to type I and type II diabetes.

The fourth aspect of the present invention proposes a method for treating or preventing diabetes in a subject comprising administering to the subject a therapeutically effective amount of the side chain compound, acylated insulin analog and pharmaceutical composition of the present invention;
the diabetes refers to type I and type II diabetes.

The fourth aspect of the present invention proposes the side chain compound, acylated insulin analog and pharmaceutical composition of the present invention for use in treating or preventing diabetes in a subject;
the diabetes refers to type I and type II diabetes.

The fifth aspect of the present invention proposes an administration method of the side chain compound, acylated insulin analog and pharmaceutical composition of the present invention, wherein the compound, the acylated insulin analog and the pharmaceutical composition are administered twice a week, once a week, or less frequently.

The sixth aspect of the present invention proposes a method for preparing a novel acylated insulin analog of formula (II), the method comprises using the side chain compound of formula (I) and human insulin analog to carry out an acylation reaction; wherein, the human insulin analog has A chain and B chain, the amino acid sequence of the A chain is shown in SEQ ID NO.1, the amino acid sequence of the B chain is shown in SEQ ID NO.2 or SEQ ID NO.3.

Compared with the prior art, the present invention has the following beneficial effects:

The present invention provides a novel acylated human insulin analog, which can be used for the treatment of diabetes, and has a longer acting time for controlling glucose compared with the current daily preparation (insulin degludec). It can be used as a weekly preparation or a longer acting insulin preparation, which can be administered subcutaneously once a week or less frequently, and will produce a satisfactory therapeutic effect for diabetic patients on the need for basal insulin therapy and improve patient compliance.

In the process of describing the present invention, the relevant terms in this article are explained and illustrated, which are only for the convenience of understanding the scheme, and should not be regarded as a limitation on the protection scheme of the present invention.

As used herein, the “insulin analog” refers to a polypeptide having a form that can be obtained by deletion and/or exchange of at least one amino acid residue present in naturally occurring insulin and/or by addition of at least one amino acid residue derived from the naturally occurring insulin, such as the molecular structure of human insulin structure.

“desB30 insulin” and “desB30 human insulin” refer to native insulin or analogs thereof lacking the B30 amino acid residue.

The term “diabetes” includes type I diabetes, type II diabetes, gestational diabetes (during pregnancy) and other conditions that cause hyperglycemia. The term is used for metabolic disorders in which the pancreas produces insufficient amounts of insulin, or in which the body's cells fail to respond appropriately to insulin, preventing cells from absorbing glucose. As a result, glucose accumulates in the blood. Type I diabetes, also known as insulin-dependent diabetes mellitus (IDDM) and juvenile-onset diabetes, is caused by B-cell destruction, often resulting in absolute insulin deficiency. Type II diabetes, also known as non-insulin-dependent diabetes mellitus (NIDDM) and adult-onset diabetes, is associated with major insulin resistance and thus relative insulin deficiency and/or major insulin secretion defect with insulin resistance.

“A14E, B16E, B25H, B29K (N(ε)-eicosanedioyl-L-Lys-succinic acid-2xOEG), desB30 human insulin” means that amino acid Y at position A14 in human insulin has been mutated to E, the amino acid Y at position B16 in human insulin has been mutated to E, the amino acid F at position B25 in human insulin has been mutated to H, the amino acid K at position B29 in human insulin has been modified by acylation with the residue eicosandioyl-L-Lys-succinic acid-2xOEG on the E nitrogen (termed N) of the lysine residue at B29, and amino acid T at position B30 in human insulin has been deleted.

“OEG” is [2-(2-aminoethoxy)ethoxy]ethylcarbonyl; 2xOEG or (OEG)₂ both refer to 2 OEGs.

“Su” is succinimidyl-1-yl=2,5-dioxo-pyrrolidin-1-yl.

“OSu” refers to succinimidyl-1-yloxy 2,5-dioxo-pyrrolidin-1-yloxy.

### DESCRIPTION OF THE DRAWINGS

FIG. 1 Changes of blood glucose in C57 mice after a single subcutaneous administration;

FIG. 2 Time and drug concentration data in J.V.PK of SD rats;

FIG. 3 Random blood glucose change curve of repeated administration to T1DM mice;

FIG. 4 Random blood glucose change curve of repeated administration to T1DM mice;

FIG. 5 Time and drug concentration curve in SC.PK of SD rats;

FIG. 6 Time and drug concentration curve in S.C.PK of C57BL6 mice;

FIG. 7 Time and drug concentration curve in J.V.PK of Beagles.

### EXAMPLES

The solution of the present invention will be explained below in conjunction with the embodiments. Examples of such embodiments are illustrated in the drawings, wherein the same or similar reference numerals refer to the same or similar components or components having the same or similar functions throughout. If no specific technique or condition is indicated in the examples, the technique or condition described in the literature in the field or the product specification is used. The reagents or instruments used without the manufacturer's indication are conventional products that can be obtained from the market. Those skilled in the art will understand that the following examples are only used to illustrate the present invention, and should not be construed as limiting the scope of the present invention.

### Example 1

Preparation of Insulin Mutant Analog (A14E, B16E, B25H, B29K, desB30 Human Insulin Analog)

Construction of vector for insulin analog, yeast expression, processing and purification can be performed using standard techniques readily recognized by those skilled in the art. A non-limiting example of the preparation of insulin analog was previously described (Glendorf T, Sorensen A R, Nishimura E, Pettersson I, & Kjeldsen T:Importance of the Solvent-Exposed Residues of the Insulin B chain α-Helix for Receptor Binding: Biochemistry. 2008; 47(16):4743-51). In short, the yeast expression system was used to connect the A and B single chains of long-acting insulin through artificially designed C-peptide, and the spacer peptide was added to increase the stability of the precursor protein and the expression of the target protein was increased. Through enzymatic cleavage and subsequent purification, both spacer peptide and C peptide were cleaved in the downstream purification process to obtain long-acting insulin analog. Complete conversion to the double-chain DesB30 analog was verified by MALDI-TOF MS, and its purity was tested by RP-HPLC under acidic and neutral conditions. The engineered strain obtained by screening the gene-transfected host bacteria can be fermented at high density, with high expression level and low fermentation cost. The designed gene facilitates the development of a simple and efficient purification process.

### 1) Construction of Recombinant Expression Vector

General Biosystems (Anhui) Co., Ltd. was entrusted to carry out the total synthesis of the target gene, and the target gene sequence and the vector pPIC9K were digested with restriction enzymes BamHI and EcoRI (TAKARA), and the digested product was purified and recovered using the Gel Extraction Kit according to the manufacturer's instructions. The vector was ligated using DNA Ligation Kit Ver2.1 (TAKARA) according to the manufacturer's instructions, and transformed into competent cells DH5a. The single colony on the plate was randomly picked, and Guangzhou Aike Biotechnology Co., Ltd. was entrusted to conduct sequencing of the target gene to verify the correctness, and then the Omega plasmid extraction kit was used to extract and verify the correct expression vector. After linearization with restriction enzyme SalI (TAKARA), the expression vector was purified and recovered with Gel Extraction Kit according to the manufacturer's instructions, and stored at −20° C. for future use.

### 2) Construction of Recombinant Engineering Strains and Protein Fermentation Expression

The above linearized recombinant expression plasmid was added to Pichia pastoris GS115 competent cells (Invitrogen), transformed by electric shock method, and the electric shock was performed with MicroPulser (Bio-Rad, 165-2100) equipment. After electric shock, 1 mL of pre-cooled 1 mol/L sorbitol was added, and the bacterial suspension was transferred to a sterilized centrifuge tube, recovered and cultured in a shaker at 30° C., 220 rpm for 2 h, then coated with MD medium plates and inverted cultured in an incubator at 30° C. The transformants grown on the plate were screened for high copy recombinants with Geneticin G418 (merck).

The above screened recombinants were cultured and fermented in a shaker flask, and a single colony was picked and inoculated into a YPD medium for cultivation, and shaken in a shaker at 30° C., 220 rpm for about 2 days, and the seed liquid obtained by cultivation was inoculated into BMGY medium (Buffered Glycerol-complex Medium) at a ratio of 1:100, incubated with shaking in a shaker at 30° C., 220 rpm for about 24 h, and then anhydrous methanol was added at 1% of the volume of the fermentation medium to induce expression of the protein, and the anhydrous methanol was supplemented every 12 h, then the fermentation was terminated after 120 h of induction. The fermentation broth was collected and centrifuged at 6000 rpm for 6 min, and the supernatant was collected. The supernatant liquid was subjected to cation chromatography, enzyme digestion, polymer chromatography, ultrafiltration, and freeze-drying. The purity of the freeze-dried sample was 90% detected by HPLC, and the molecular weight was detected by MALDI-TOF MS. The detection value of molecular weight of A14E, B16E, B25H, Des(B30) human insulin analog was 5628.41 Da, and the theoretical value was 5628.39 Da, the detection value was consistent with the theoretical value; the detection value of molecular weight of A14E, B16H, B25H, Des(B30) human insulin analog was 5637.06 Da, the theoretical value was 5636.31 Da, the detection value was consistent with the theoretical value.

### Example 2 Preparation of Long-Acting Insulin

### 2.1 Preparation of A14E, B16E, B25H, B29K(N(ε)-COOH(CH₂)₁₈CO-L-Lys-CO(C H₂)₂CO—(OEG)₂), desB30 human insulin analog

### (1) Preparation Process of COOH(CH₂)₁₈CO-L-Lys-CO (CH₂)₂CO—(OEG)₂-OSu Side Chain Compound

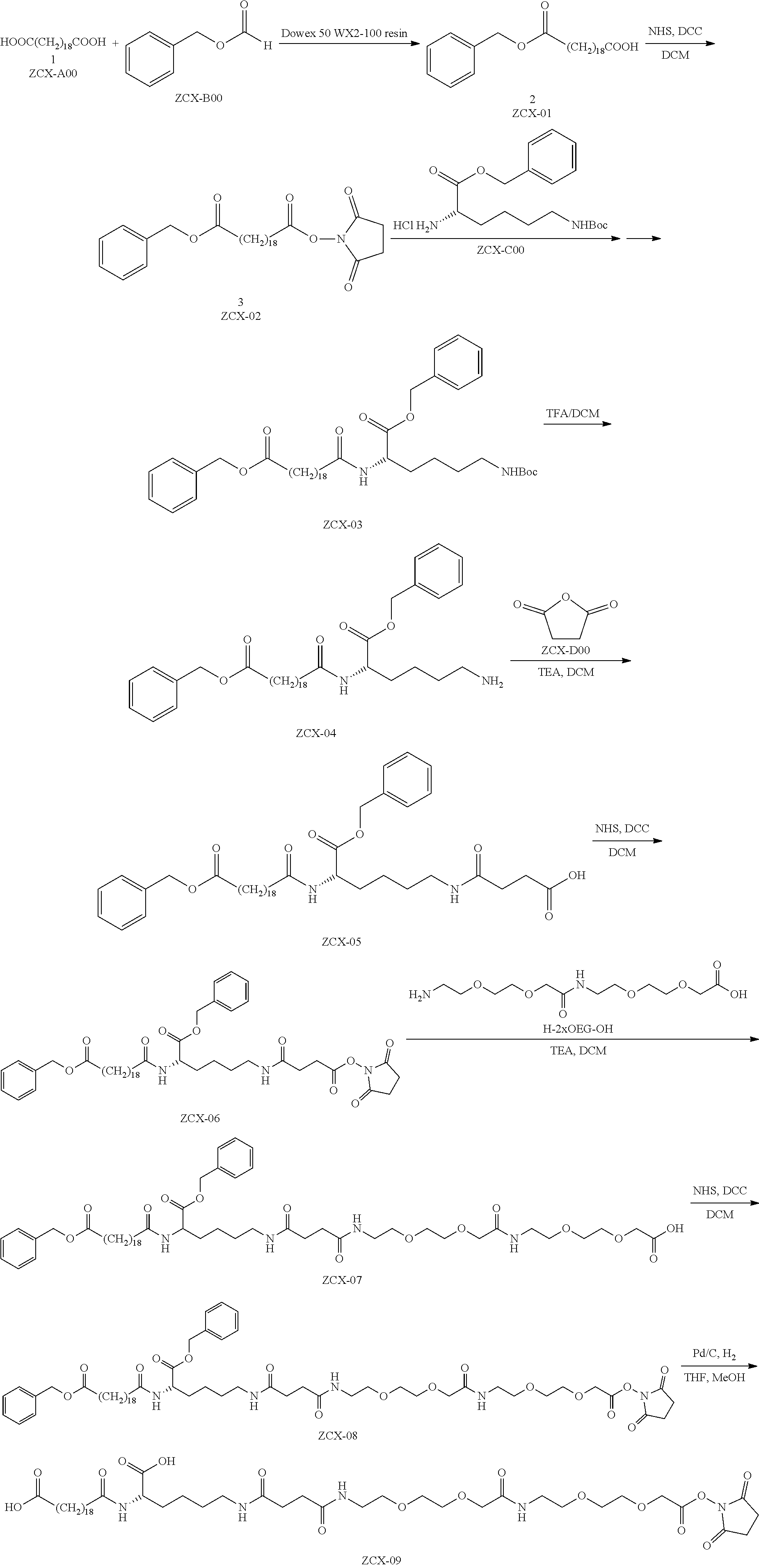

a) ZCX-A00 (40 g, 58.39 mmol), ZCX—B00 (31.80 g, 233.56 mmol), Dowex50 WX2-100 acidic cationic resin (60 g) and 360 mL of n-octane were added to a three-neck round bottom flask, the mixture was stirred and kept at reflux for 72 h after the temperature was raised to 110° C. The heating power was turned off, the mixture was kept stirring and returned to room temperature. The filtrate was discarded by suction filtration to obtain filter residue, then 360 mL of dichloromethane was added to the filter residue and stirred at room temperature for 2 h, and then the filter residue was discarded by suction filtration, the obtained filtrate was concentrated to dryness in vacuo to obtain a solid crude product. 60 mL of isopropanol was added to the solid crude product to recrystallize and 16.19 g of product ZCX-01 was obtained.

ESI-MS m/z: 433.33[M+H]⁺, which was consistent with the theoretical value.

b) ZCX-01 (10.0 g, 23.11 mmol) and 130 mL of dichloromethane were added to a 250 mL single-neck flask, then N-hydroxysuccinimide (2.93 g, 25.42 mmol) and dicyclohexylcarbodiimide (5.72 g, 27.73 mmol) were added, the mixture was reacted at 30° C. for 24 h. Then the mixture was filtered to remove the precipitate, distilled and concentrated to dryness to obtain a solid crude product. 60 mL of isopropanol and 60 mL of n-heptane were added to the solid crude product to recrystallize and 10.15 g of product ZCX-02 was obtained.

ESI-MS m/z: 530.32[M+H]⁺, which was consistent with the theoretical value.

c) ZCX-02 (10.6 g, 20 mmol), ZCX—C00 (lysine derivative, 8.2 g, 22 mmol) and 150 mL of dichloromethane were added into a 250 mL single-neck round bottom flask, the mixture was stirred at room temperature, then 5.5 mL of triethylamine was added. 2N hydrochloric acid solution was added to the mixture to adjust the pH=1-2, the mixture was kept stirring for 30 min, then separated, the aqueous phase was discarded, the organic phase was concentrated to dryness in vacuo, and purified by column chromatography to obtain the product Na-(long aliphatic chain diacid)-L-Lys-1-benzyl ester-6-Boc.

ESI-MS m/z: 752.52[M+H]⁺, which was consistent with the theoretical value.

d) The H NMR data of ZCX-03 showed that the obtained structure was the target product ZCX-03.

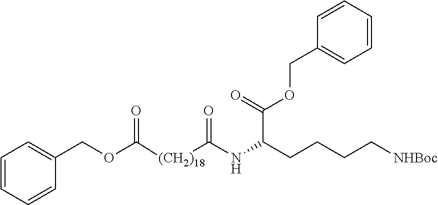

1H-NMR (400 MHz, CDCl3) δ 7.37 (s, 10H), 6.08 (d, J 7.2 Hz, 1H), 5.19 (dd, J 26.8, 13.8 Hz, 4H), 4.66 (dd, J=12.5, 7.4 Hz, 1H), 4.54 (s, 1H), 3.07 (d, J=6.0 Hz, 2H), 2.37 (t, J=7.5 Hz, 2H), 2.29-2.17 (m, 2H), 1.87 (d, J=34.8 Hz, 1H), 1.73 (d, J=14.2 Hz, 1H), 1.68-1.58 (m, 4H), 1.46 (s, 11H), 1.28 (d, J=12.9 Hz, 30H).

ZCX-03 (11.5 g, 15 mmol), 55 mL of trifluoroacetic acid and 55 mL of dichloromethane were added to a single-neck round-bottomed flask, then the flask was placed in a 0° C. low temperature tank and the mixture was stirred and reacted for 1 h. After the reaction was basically complete by TLC detection, the reaction system was concentrated in vacuo to dryness to obtain viscous liquid, then 200 mL of dichloromethane was added to dissolve, the mixture was washed with saturated NaHCO₃ solution, then separated, the organic phase was washed twice with saturated brine, separated, the organic phase was concentrated to dryness in vacuo, recrystallized with anhydrous ethanol, and 8.35 g of product ZCX—C04 was obtained.

ESI-MS m/z 651.56[M+H]⁺, which was consistent with the theoretical value.

e) ZCX—C04 (8.00 g, 12.31 mmol), l50 mL of dichloromethane and 3 mL of triethylamine were added into a single-neck round-bottomed flask, the mixture was stirred to dissolve at room temperature, then succinic anhydride (2.46 g, 24.62 mmol) was added. After the addition, the mixture was stirred and reacted at 30° C. for 24 h. After the reaction was basically complete by TLC detection, 10 mL of 2N HCl solution was added to adjust the pH=1-2, then the mixture was stirred for 30 min and separated, the aqueous phase was discarded, the organic phase was washed once with saturated brine, then separated, the organic phase was dried over anhydrous Na₂SO₄, filtered, and the filtrate was concentrated to dryness in vacuo to obtain a crude solid product. The crude solid product was recrystallized with anhydrous ethanol, and 8.9 g of product ZCX—C05 was obtained.

ESI-MS m/z 751.03[M+H]⁺, which was consistent with the theoretical value.

The H NMR data of ZCX-05 showed that the obtained structure was the target product ZCX-05.

1H NMR (400 MHz, CDCl3) δ 7.38 (d, J 3.7 Hz, 10H), 6.39-6.26 (m, 2H), 5.19 (q, J=12.2 Hz, 2H), 5.13 (s, 2H), 4.66 (td, J=8.4, 4.6 Hz, 1H), 3.24 (d, J=59.8 Hz, 2H), 2.74-2.65 (m, 2H), 2.51 (dd, J=10.4, 5.6 Hz, 2H), 2.37 (t, J=7.5 Hz, 2H), 2.30-2.23 (m, 2H), 1.85 (d, J=34.4 Hz, 1H), 1.65 (d, J=24.7 Hz, 5H), 1.52 (d, J=36.5 Hz, 2H), 1.38-1.22 (m, 30H).

f) ZCX-05 (8.5 g, 11.32 mmol) and 130 mL of dichloromethane were added to a 250 mL single-neck flask, then N-hydroxysuccinimide (1.95 g, 16.98 mmol) and dicyclohexylcarbodiimide (3.50 g, 16.98 mmol) were added. The mixture was continuously reacted at 30° C. for 24 h, then filtered to remove the precipitate, distilled and concentrated to dryness to obtain a crude solid product ZCX-06, which was directly used in the next step without purification.

To the solid crude product ZCX-06 obtained in the previous step were added [2-(2-{2-[2-(2-aminoethoxy)ethoxy]acetylaminolethoxy)ethoxy]acetic acid (the alternative name is H-2xOEG-OH) (3.73 g, 11.32 mmol) and 130 mL of dichloromethane, the mixture was stirred at room temperature for 10 min, then 2.4 mL of triethylamine was added. After the addition, the mixture was stirred and reacted at 30° C. for 24 h. After the reaction was basically complete by TLC detection, 10 mL of 2N HCl solution was added to the mixture and stirred for 30 min, then separated, the aqueous phase was discarded, the organic phase was washed twice with saturated brine, separated, the aqueous phase was discarded, the organic phase was dried over anhydrous Na₂SO₄, filtered, the filtrate was concentrated to dryness in vacuo to obtain a crude solid product. The crude solid product was purified by column chromatography to obtain 5.50 g of product ZCX-07.

ESI-MS m/z 1042.59[M+H]⁺, which was consistent with the theoretical value.

g) ZCX-07 (5.00 g, 4.80 mmol) and 130 mL of dichloromethane were added to a 250 mL one-neck flask, then N-hydroxysuccinimide (0.83 g, 7.2 mmol) and dicyclohexylcarbodiimide (1.49 g, 7.2 mmol) were added. The mixture was continuously reacted at 30° C. for 24 h, then filtered to remove the precipitate, distilled and concentrated to dryness to obtain a solid crude product. To the solid crude product were added 50 mL of isopropanol and 50 mL of n-heptane to recrystallize, and 4.30 g of product ZCX-08 was obtained.

ESI-MS m/z 1139.10[M+H]⁺, which was consistent with the theoretical value.

The H NMR data of ZCX-08 showed that the obtained structure was the target product ZCX-08.

1H NMR (400 MHz, CDCl3) δ 7.36 (s, 10H), 7.27-7.22 (m, 1H), 6.63 (s, 1H), 6.29 (dd, J=11.9, 6.7 Hz, 2H), 5.18 (t, J=9.8 Hz, 2H), 5.12 (s, 2H), 4.61 (td, J=7.9, 5.1 Hz, 1H), 4.51 (s, 2H), 4.02 (s, 2H), 3.83-3.76 (m, 2H), 3.70-3.66 (m, 4H), 3.61 (dd, J=8.7, 4.0 Hz, 4H), 3.56-3.48 (m, 4H), 3.47-3.40 (m, 2H), 3.23-3.12 (m, 2H), 2.87 (s, 4H), 2.52 (d, J=5.2 Hz, 2H), 2.47 (d, J=5.4 Hz, 2H), 2.36 (t, J=7.5 Hz, 2H), 2.23 (t, J=7.6 Hz, 2H), 1.80 (s, 1H), 1.64 (dd, J=14.5, 7.3 Hz, 5H), 1.53-1.44 (m, 2H).

h) ZCX-08 (1.40 g, 1.22 mmol), 10% Pd/C (0.12 g), 0.1 mL of trifluoroacetic acid, 30 mL of THF and 10 mL of methanol were added into a single-neck round bottom flask, the flask was replaced with hydrogen 3 times and sealed with a hydrogen balloon, the mixture was placed at 30° C. and stirred for 6 h for hydrogenation and debenzylation. After the reaction was basically complete by TLC detection, the mixture was filtered to remove 10% Pd/C, 120 mL of n-heptane was added dropwise to the organic filtrate and kept stirring. During the dropwise addition, solid was precipitated, and after the dropping was completed, the mixture was stirred at room temperature for 0.5 h, filtered to obtain 0.83 g of product ZCX-09, which was COOH(CH₂)₁₈CO-L-Lys-CO (CH₂)₂CO—(OEG)₂-OSu aliphatic side chain.

ESI-MS m/z 959.45[M+H]⁺, which was consistent with the theoretical value.

The H NMR data of ZCX-09 showed that the obtained structure was the target product ZCX-09.

1H NMR (400 MHz, DMSO) δ 4.63 (d, J 25.1 Hz, 2H), 3.88 (s, 2H), 3.28 (dd, J 11.5, 5.7 Hz, 2H), 3.19 (dd, J=11.3, 5.6 Hz, 2H), 2.83 (s, 3H), 2.22-2.14 (m, 2H), 2.10 (t, J=7.3 Hz, 2H), 1.60-1.42 (m, 4H), 1.26 (d, J=24.8 Hz, 26H).

### (2) Preparation of A14E,B16E,B25H,B29K(N(ε)-COOH(CH₂)₁CO-L-Lys-CO(CH₂)₂CO—(OEG)₂), desB30 Human Insulin Analog

A14E, B16E, B25H, Des (B30) human insulin (60 mg, 0.01 mmol) was dissolved in a solution of 5 mL pure water and 2 mL DMF, the mixture was placed in a 10° C. low temperature reaction bath, and then 100 ul of triethylamine was added dropwise to adjust the pH to 11.50. COOH(CH₂)₁₈CO-L-Lys-CO(CH₂)₂CO—(OEG)₂-OSu side chain compound (14.37 mg, 0.015 mmol) was dissolved in 3 mL DMF to form a side chain mixed solution, under stirring, the side chain mixed solution was quickly added to the above reaction system, and 1N NaOH solution was used to keep the pH of the reaction system constant at 11.00-11.50. After the addition, the timing was started, after 1.0 h of reaction, the pH of the solution was adjusted to 7.0-7.5 with 1N HCl solution. The reaction was terminated to obtain the crude product solution of the acylation of reactive protein, the reaction process was controlled by RP-HPLC.

### (3) Purification of A14E,B16E,B25H,B29K(N(ε)-COOH(CH₂)₁₈CO-L-Lys-CO(CH₂)₂CO—(OEG)₂), desB30 Human Insulin Analog

The above protein acylation crude product solution was diluted with water to make the organic phase content about 15% (v:v), filtered with a 0.45 μm filter membrane, and then purified by RP-HPLC to obtain a purified solution.

### (4) Ultrafiltration and lyophilization of A14E,B16E,B25H,B29K(N(ε)-COOH(CH₂)₁₈CO-L-Lys-CO(CH₂)₂CO—(OEG)₂), desB30 Human Insulin Analog

The above purified solution was replaced with water for injection using an ultrafiltration membrane package system, then freeze-dried to obtain 23 mg of a lyophilized product, the molecular structure of the obtained human insulin analog was as follows:

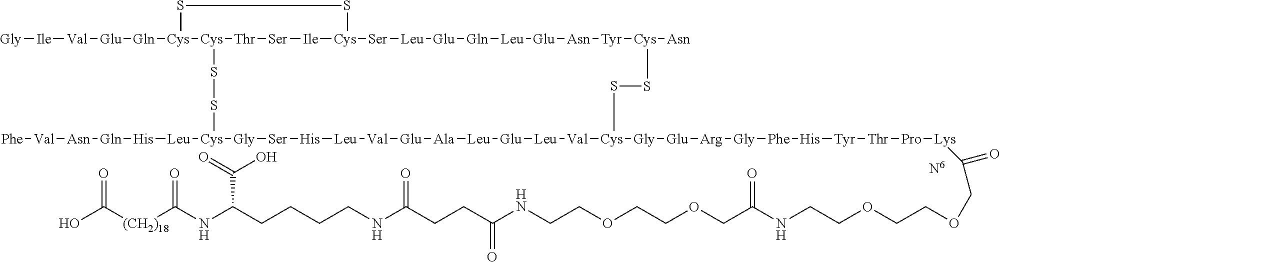

### (5) Structural Confirmation of A14E,B16E,B25H,B29K(N(ε)-COOH(CH₂)₁₈CO-L-Lys-C O(CH₂)₂CO—(OEG)₂), desB30 Human Insulin Analog

The measured mass spectrum of A14E, B16E, B25H, B29K(N(ε)-COOH(CH₂)₁₈CO-L-Lys-CO(CH₂)₂CO—(OEG)₂), desB30 human insulin analog was 6471.42 Da, which was consistent with the theoretical molecular weight of 6471.64 Da. It was showed that A14E, B16E, B25H, B29K(N(ε)-COOH(CH₂)₁₈CO-L-Lys-CO(CH₂)₂CO—(OEG)₂), desB30 human insulin analog was successfully prepared, which can be abbreviated as Insulin-a3.

### 2.2 Preparation of A14E, B16E, B25H, B29K(N(ε)-COOH(CH₂)₁₆CO-L-Lys-CO(C H₂)₂CO—(OEG)₂), desB30 Human Insulin Analog

### (1) Preparation Process of COOH(CH₂)₁₆CO-L-Lys-CO(CH₂)₂CO—(OEG)₂-OSu Side Chain Compound

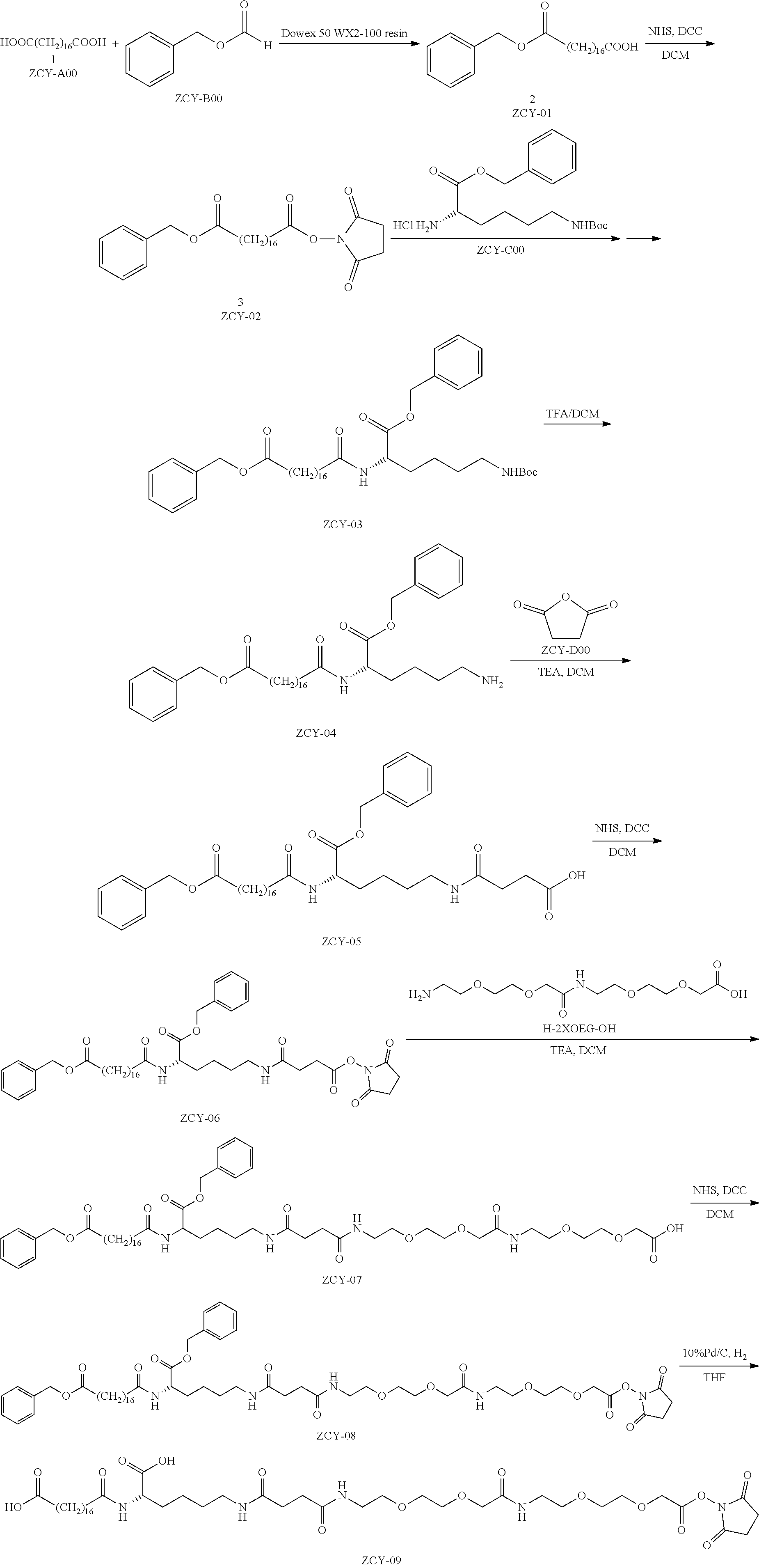

The preparation method of COOH(CH₂)₁₆CO-L-Lys-CO (CH₂)₂CO—(OEG)₂-OSu side chain (referred to as ZCY-09) is similar to the preparation method of the COOH(CH₂)₁₈CO-L-Lys-CO(CH₂)₂CO—(OEG)₂-OSu side chain compound in Example 2.1, the structure and MS test of the prepared target product are shown below.

ESI-MS m/z 931.40[M+H]⁺, which was consistent with the theoretical value.

The H NMR data of ZCY-09 showed that the obtained structure was the target product ZCY-09.

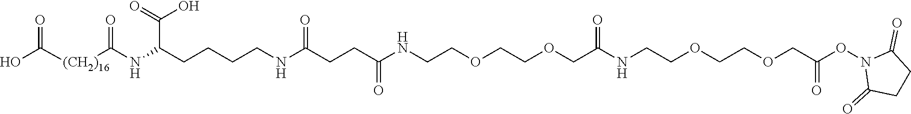

1H NMR (400 MHz, DMSO) δ 4.60 (s, 2H), 3.85 (d, J 26.1 Hz, 2H), 2.83 (d, J 4.1 Hz, 5H), 2.28 (p, J=7.9 Hz, 4H), 2.18 (t, J=7.3 Hz, 2H), 2.10 (t, J=7.3 Hz, 2H).

### (2) Preparation of A14E, B16E, B25H, B29K(N(ε)-COOH(CH₂)₁₆CO-L-Lys-CO(CH₂)₂ CO—(OEG)₂), desB30 Human Insulin Analog

A14E, B16E, B25H, Des (B30) human insulin (60 mg, 0.01 mmol) was dissolved in a solution of 5 mL pure water and 2 mL DMF, the mixture was placed in a 10° C. low temperature reaction bath, and then 100 ul of triethylamine was added dropwise to adjust the pH to 11.50. COOH(CH₂)₁₆CO-L-Lys-CO (CH₂)₂CO—(OEG)₂-OSu side chain (13.95 mg, 0.015 mmol) was dissolved in 3 mL DMF to form a side chain mixed solution. Under stirring, the side chain mixed solution was quickly added to the above reaction system, and 1N NaOH solution was used to keep the pH of the reaction system constant at 11.00-11.50. After the addition, the timing was started, after 1.0 h of reaction, the pH of the solution was adjusted to 7.0-7.5 with 1N HCl solution. The reaction was terminated to obtain the crude product solution of the acylation of reactive protein, the reaction process was controlled by RP-HPLC.

### (3) Purification of A14E, B16E, B25H, B29K(N(ε)-COOH(CH₂)₁₆CO-L-Lys-CO(CH₂)₂ CO—(OEG)₂), desB30 Human Insulin Analog

The above protein acylation crude product solution was diluted with water to make the organic phase content about 15% (v:v), filtered with a 0.45 μm filter membrane, and then purified by RP-HPLC to obtain a purified solution.

### (4) Ultrafiltration and Lyophilization of A14E, B16E, B25H, B29K(N(ε)-COOH(CH₂)₁₆ CO-L-Lys-CO(CH₂)₂CO—(OEG)₂), desB30 Human Insulin Analog

The above purified solution was replaced with water for injection using an ultrafiltration membrane package system, then freeze-dried to obtain 18 mg of a lyophilized product, the molecular structure of the obtained human insulin analog was as follows:

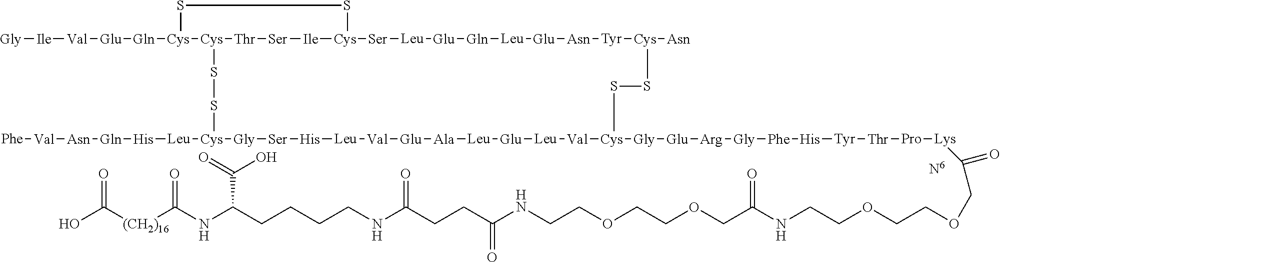

### (5) Structural Confirmation of A14E, B16E, B25H, B29K(N(ε)-COOH(CH₂)₁₆CO-L-Lys-CO(CH₂)₂CO—(OEG)₂), desB30 Human Insulin Analog

The measured mass spectrum of A14E, B16E, B25H, B29K(N(ε)-COOH(CH₂)₁₆CO-L-Lys-CO(CH₂)₂CO—(OEG)₂), desB30 human insulin analog was 6443.40 Da, which was consistent with the theoretical molecular weight of 6443.41 Da. It was showed that A14E, B16E, B25H, B29K(N(ε)-COOH(CH₂)₁₆CO-L-Lys-CO(CH₂)₂CO—(OEG)₂), desB30 human insulin analog was successfully prepared, which can be abbreviated as Insulin-a2.

### 2.3 Preparation of A14E, B16H, B25H, B29K(N(ε)-COOH(CH₂)₁₈CO-L-Lys-CO(C H₂)₂CO—(OEG)₂), desB30 Human Insulin Analog

The preparation method of COOH(CH₂)₁₈CO-L-Lys-CO (CH₂)₂CO—(OEG)₂-OSu side chain is the same as the preparation method of the COOH(CH₂)₁₈CO-L-Lys-CO(CH₂)₂CO—(OEG)₂-OSu side chain compound in Example 2.1.

A14E, B16H, B25H, Des(B30) human insulin (60 mg, 0.01 mmol) was dissolved in a solution of 5 mL pure water and 2 mL DMF, the mixture was placed in a 10° C. low temperature reaction bath, and then 100 μL of triethylamine was added dropwise to adjust the pH to 11.50. Nα-(Eicosandioic acid)-Nε—(OCCH₂CH₂CO-(2xOEG-OSu)-L-Lys side chain (14.37 mg, 0.015 mmol) was dissolved in 3 mL DMF to form a side chain mixed solution. The side chain mixed solution was quickly added to the above reaction system under stirring, and 1N NaOH solution was used to keep the pH of the reaction system constant at 11.00-11.50. After the addition, the timing was started. After 1.0 h of reaction, the pH of the solution was adjusted to 7.0-7.5 with 1N HCl solution. The reaction was terminated to obtain the crude product solution of the acylation of reactive protein, the reaction process was controlled by RP-HPLC.

The above protein acylation crude product solution was diluted with water to make the organic phase content about 15% (v:v), filtered with a 0.45 μm filter membrane, and then purified by RP-HPLC to obtain a purified solution.

The above purified solution was replaced with water for injection using an ultrafiltration membrane package system, then freeze-dried to obtain 16 mg of a lyophilized product, the molecular structure of the obtained human insulin analog was as follows:

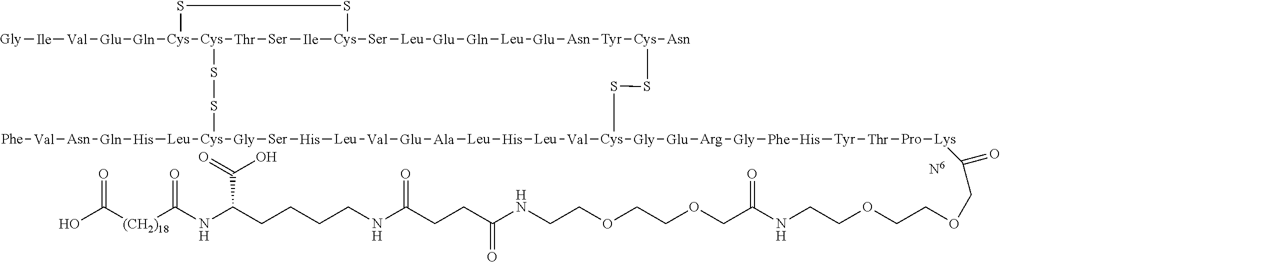

The measured mass spectrum of A14E, B16H, B25H, B29K(N(ε)-COOH(CH₂)₁₈CO-L-Lys-CO(CH₂)₂CO—(OEG)₂), desB30 human insulin analog was 6480.10 Da, which was consistent with the theoretical molecular weight of 6480.10 Da. It was showed that A14E, B16H, B25H, B29K(N(ε)-COOH(CH₂)₁₈CO-L-Lys-CO(CH₂)₂CO—(OEG)₂), desB30 human insulin analog was successfully prepared, which can be abbreviated as Insulin-a10.

### 2.4 Preparation of A14E, B16E, B25H, B29K (N^{ε}-eicosanedioyl-gGlu-2xOEG), DesB30 Human Insulin Analog

### (1) Preparation of 19-((S)-1-tert-butoxycarbonyl-3-{2-[2-({2-[2-(2,5-dioxo-pyrrolidin-1-yl oxycarbonylmethoxy)ethoxy]ethylcarbamoyl}methoxy)ethoxy]ethylcarbamoyl}propylcarbamoyl) nonadecanoic acid tert-butyl ester

(the alternative name is ^{t}Bu-eicosandioyl-gGlu)(O^{t}Bu)-2xOEG-Osu)

TSTU (1.50 g) and DIPEA (0.91 mL) were added to a solution containing 19-((S)-1-tert-butoxycarbonyl-3-{2-[2-({2-[2-(2,5-dioxo-pyrrolidin-1-yloxycarbonylmethoxy)eth oxy]ethylcarbamoyl}methoxy)ethoxy]ethylcarbamoyl}propylcarbamoyl)nonadecanoic acid tert-butyl ester (3.0 g, purchased from Shanghai Topbiochem Technology Co., Ltd.) in acetonitrile (60 ml), and the mixture was stirred overnight at room temperature, then concentrated in vacuo. Aqueous 0.1 N HCl (100 mL) and ethyl acetate (200 mL) were added to the residue, then separated, the aqueous phase was extracted with ethyl acetate (50 mL), the organic phases were combined and washed once with saturated brine, dried over anhydrous magnesium sulfate and concentrated in vacuo to obtain 3.21 g of oily liquid.

ESI-MS m/z 972.30[M+H]⁺, which was consistent with the theoretical value.

### (2) 19-((S)-1-carboxy-3-12-[2-({2-[2-(2,5-dioxo-pyrrolidin-1-yloxycarbonylmethoxy)ethoxy]ethylcarbamoyl}methoxy)ethoxy]ethylcarbamoyl}propylcarbamoyl)nonadecanoic acid

(the alternative name is eicosandioyl-gGlu-2xOEG-OSu)

^{t}Bu-eicosandioyl-gGlu)(O^{t}Bu)-2xOEG-Osu (3.0 g) was added to trifluoroacetate (66 mL) and the mixture was stirred at room temperature for 45 min. After the reaction was complete by TLC detection, the mixture was concentrated in vacuo to obtain oily liquid, then concentrated 3 times with toluene to obtain a solid. Isopropyl alcohol was used to recrystallize and filter to obtain 2.35 g of a white solid.

ESI-MS m/z 860.60[M+H]⁺, which was consistent with the theoretical value.

### (3) Preparation of (A14E, B16E, B25H, B29K (N^{ε}-eicosanedioyl-gGlu-2xOEG), DesB30 Human Insulin Analog

A14E, B16E, B25H, Des(B30) human insulin (60 mg, 0.01 mmol) was dissolved in a solution of 5 ml pure water and 2 mL DMF, the mixture was placed in a 10° C. low temperature reaction bath, and then 100 ul of triethylamine was added dropwise to adjust the pH to 11.50. Eicosandioyl-gGlu-2xOEG-OSu aliphatic side chain (15.00 mg, 0.017 mmol) was dissolved in 3 mL DMF to form a side chain mixed solution, under stirring, the side chain mixed solution was quickly added to the above reaction system, and 1N NaOH solution was used to keep the pH of the reaction system constant at 11.00-11.50. After the addition, the timing was started, after 1.0 h of reaction, the pH of the solution was adjusted to 7.0-7.5 with 1N HCl solution. The reaction was terminated to obtain the crude product solution of the acylation of reactive protein, the reaction process was controlled by RP-HPLC.

### (4) Purification of A14E, B16E, B25H, B29K (N^{ε}-eicosanedioyl-gGlu-2xOEG), DesB30 Human Insulin Analog

The above protein acylation crude product solution was diluted with water to make the organic phase content about 15% (v:v), filtered with a 0.45 μm filter membrane, and then purified by RP-HPLC to obtain a purified solution.

### (5) Ultrafiltration and lyophilization of A14E, B16E, B25H, B29K (N^{ε}-eicosanedioyl-Glu-2xOEG), DesB30 human insulin analog

The above purified solution was replaced with water for injection using an ultrafiltration membrane package system, then freeze-dried to obtain 9.3 mg of a lyophilized product, the molecular structure of the obtained human insulin analog was as follows:

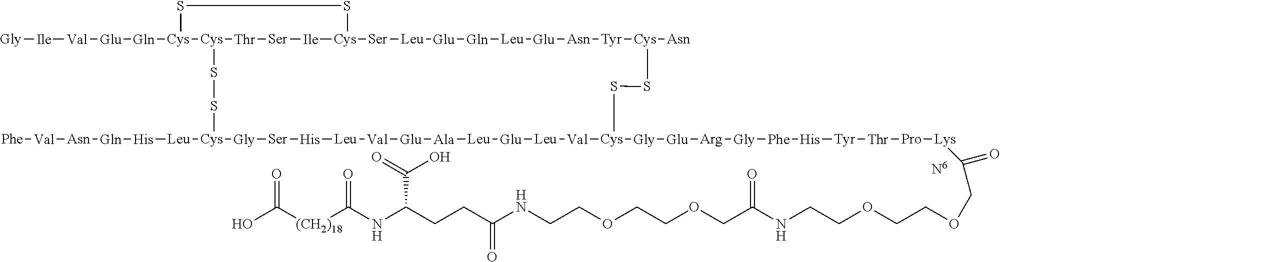

### (6) Structural Confirmation of A14E, B16E, B25H, B29K (N^{ε}-eicosanedioyl-Glu-2xOEG), DesB30 Human Insulin Analog

The measured mass spectrum of A14E, B16E, B25H, B29K (N^{ε}-eicosanedioyl-gGlu-2xOEG), DesB30 human insulin analog was 6372.28 Da, which was consistent with the theoretical molecular weight of 6372.33 Da. It was showed that the target product of A14E, B16E, B25H, B29K (N^{ε}-eicosanedioyl-gGlu-2xOEG), DesB30 human insulin analog was successfully prepared, which can be abbreviated as Insulin-a1.

### 2.5 Preparation of A14E, B16H, B25H, B29K (N^{ε}-eicosanedioyl-gGlu-2xOEG), DesB30 Human Insulin Analog

### (1) Preparation of 19-((S)-1-tert-butoxycarbonyl-3-{2-[2-({2-[2-(2,5-dioxo-pyrrolidin-1-yl oxycarbonylmethoxy)ethoxy]ethylcarbamoyl}methoxy)ethoxy]ethylcarbamoyl}propylcarbamoyl) nonadecanoic acid tert-butyl ester

(the alternative name is ^{t}Bu-eicosandioyl-gGlu)(O^{t}Bu)-2xOEG-Osu)

TSTU (1.50 g) and DIPEA (0.91 mL) were added to a solution containing 19-((S)-1-tert-butoxycarbonyl-3-12-[2-({2-[2-(2,5-dioxo-pyrrolidin-1-yloxycarbonylmethoxy)eth oxy]ethylcarbamoyl}methoxy)ethoxy]ethylcarbamoyl}propylcarbamoyl)nonadecanoic acid tert-butyl ester (3.0 g, purchased from Shanghai Topbiochem Technology Co., Ltd.) in acetonitrile (60 mL), and the mixture was stirred overnight at room temperature, then concentrated in vacuo. Aqueous 0.1 N HCl (100 mL) and ethyl acetate (200 mL) were added to the residue, then separated, the aqueous phase was extracted with ethyl acetate (50 mL), the organic phases were combined and washed once with saturated brine, dried over anhydrous magnesium sulfate and concentrated in vacuo to obtain 3.21 g of oily liquid.

ESI-MS m/z 972.30[M+H]⁺, which was consistent with the theoretical value.

### (2) 19-((S)-1-carboxy-3-12-[2-({2-[2-(2,5-dioxo-pyrrolidin-1-yloxycarbonylmethoxy)ethoxy]ethylcarbamoyl}methoxy)ethoxy]ethylcarbamoyl}propylcarbamoyl)nonadecanoic acid

(the alternative name is eicosandioyl-gGlu-2xOEG-OSu)

^{t}Bu-eicosandioyl-gGlu)(O^{t}Bu)-2xOEG-Osu (3.0 g) was added to trifluoroacetate (66 mL) and the mixture was stirred at room temperature for 45 min. After the reaction was complete by TLC detection, the mixture was concentrated in vacuo to obtain oily liquid, then concentrated 3 times with toluene to obtain a solid. Isopropyl alcohol was used to recrystallize and filter to obtain 2.35 g of a white solid.

ESI-MS m/z 860.60[M+H]⁺, which was consistent with the theoretical value.

### (3) Preparation of (A14E, B16H, B25H, B29K (N^{ε}-eicosanedioyl-Glu-2xOEG), DesB30 Human Insulin Analog

A14E, B16H, B25H, Des(B30) human insulin (60 mg, 0.01 mmol) was dissolved in a solution of 5 mL pure water and 2 mL DMF, the mixture was placed in a 10° C. low temperature reaction bath, and then 100 μL of triethylamine was added dropwise to adjust the pH to 11.50. Eicosandioyl-gGlu-2xOEG-OSu aliphatic side chain (15.00 mg, 0.017 mmol) was dissolved in 3 mL DMF to form a side chain mixed solution, under stirring, the side chain mixed solution was quickly added to the above reaction system, and 1N NaOH solution was used to keep the pH of the reaction system constant at 11.00-11.50. After the addition, the timing was started, after 1.0 h of reaction, the pH of the solution was adjusted to 7.0-7.5 with 1N HCl solution. The reaction was terminated to obtain the crude product solution of the acylation of reactive protein, the reaction process was controlled by RP-HPLC.

The above protein acylation crude product solution was diluted with water to make the organic phase content about 15% (v:v), filtered with a 0.45 μm filter membrane.

The above purified solution was replaced with water for injection using an ultrafiltration membrane package system, then freeze-dried to obtain 13.21 mg of a lyophilized product, the molecular structure of the obtained human insulin analog was as follows:

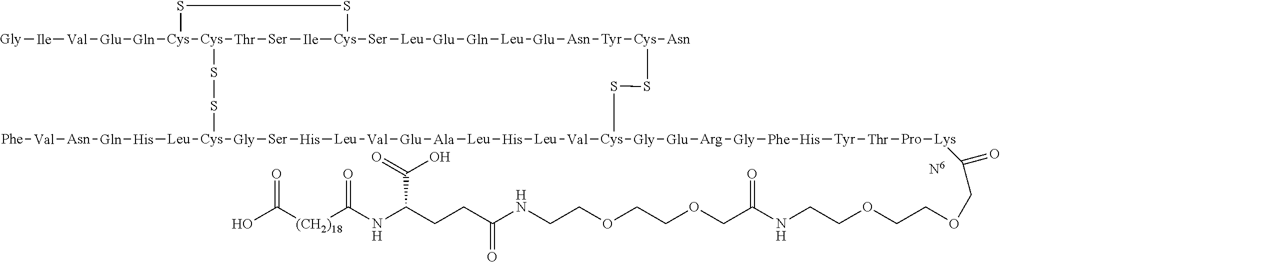

The measured mass spectrum of A14E, B16H, B25H, B29K (N^{ε}-eicosandioyl-gGlu-2xOEG), DesB30 human insulin analog was 6381.01 Da, which was consistent with the theoretical molecular weight of 6381.51 Da. It was showed that the target product of A14E, B16H, B25H, B29K (N^{ε}-eicosandioyl-gGlu-2xOEG), DesB30 human insulin analog was successfully prepared, which can be abbreviated as Icodec.

### Example 3 In Vitro Biological Activity Test of Insulin

The acylated insulin analog of the present invention can activate the cells transfected with insulin receptor B to generate insulin receptor autophosphorylation, and can also reversibly bind to human serum albumin (HSA). The phosphorylation level of insulin receptor B was detected by Cisbio's Phospho-IR beta (Tyr1150/1151) kit method to evaluate the biological activity of insulin. The cells were seeded into a 96-well plate overnight, and after the serum in the medium was removed, 40 μl of serum-free medium was added to culture for about 4 h. Then a dilution series of insulin derivatives were prepared with blank solution (0.6% casein, 0.06 mg/mL EDTA, 1×DPBS) and incubated with cells in the 96-well plate for 5 min in a CO₂ incubator (37° C., 5% CO₂). The liquid in the 96-well plate was poured off, 100 L of a mixture of lysis buffer (2% Triton X-100, 150 mM NaCl, 50 mM HEPES, pH=7) and inhibitor (Blocking Reagent in the kit) was added to lyse the cells, then the plate was shaken at 350 rpm for 30 min. Relative activity (in percent (%)) was assessed by measuring insulin receptor phosphorylation levels in the supernatant after cell lysis and fitting a curve to the data using nonlinear regression in Graphpad Prism 5 software. Related assays were also used, in which the blank solution also contained 1.5% HSA to simulate physiological conditions. Changes in the phosphorylation levels of the insulin-activated insulin receptors of the invention were detected as an indirect reflection of the albumin binding activity.

TABLE 1
In vitro activity data of insulin analog

| | 0% HSA | | 1.5% HSA | | |
|---|---|---|---|---|---|
| | | Relative | | Relative | Relative |
| | EC50 | HI | EC50 | HI | 0% HSA |
| Sample lot | (nM) | activity | (nM) | activity | activity |
| human | 1.232 | 1 | 2.592 | 1 | 48% |
| insulin (HI) | | | | | |
| Degludec | 4.551 | 27.07% | 20.59 | 12.59% | 22.10% |
| (DEG.) | | | | | |
| Icodec | 77.20 | 1.60% | 1477 | 0.17% | 5.23% |
| Insulin-a1 | 230 | 0.54% | 5017 | 0.05% | 4.58% |
| insulin-a3 | 213.2 | 0.58% | 4488 | 0.06% | 5.86% |
| Insulin-a10 | 85.47 | 1.44% | 1310 | 0.20% | 6.52% |

Remarks: (1) A14E, B16E, B25H, B29K (N^{ε}-eicosanedioyl-gGlu-2xOEG), DesB30 human insulin analog, the compound is abbreviated as Insulin-a1.

(2) A14E, B16H, B25H, B29K (N^{ε}-eicosanedioyl-gGlu-2xOEG), DesB30 human insulin analog, the compound is abbreviated as Icodec.

From the data in Table 1, it can be seen that the in vitro activities of the new fatty side chain acylation to prepare new insulin drugs insulin-a3 and insulin-a10 are significantly decreased compared with recombinant human insulin or insulin degludec under the conditions of 0% HSA and 1.5% HAS. The main reason is that the binding of the side chain to albumin is stronger, and the binding of insulin precursor to the receptor is weaker, so the side chain has a certain effect on the in vitro activity, and it also reveals the different binding abilities of the new insulin drug and albumin. In this repeated administration experiment of C57BL6 mice modeled by STZ, it can be seen that the effective glucose control effect of control Insulin-a1 can be maintained for 3 days/time, and the effective glucose control effect of Insulin-a3 can be maintained for 4-5 days/time. It can be seen that Insulin-a3 has a longer glucose control maintenance time than the control Insulin-a1, and the effect is better. Therefore, when the insulin precursors are the same, this reversible binding force is better in new insulin drugs.

### Example 4 Hypoglycemic Effect of Test Drugs on Normal C57BL/6 Mice

### (1) Test Product

TABLE 2

| Name | Supplier | Physical state | Storage conditions |
|---|---|---|---|
| Degludec | Dongguan HEC | Colorless liquid | 4° C. |
| Icodec | Biopharmaceutical | Colorless liquid | 4° C. |
| Insulin-a1 | R&D Co., Ltd. | Colorless liquid | 4° C. |
| Insulin-a3 | | Colorless liquid | 4° C. |
| Insulin-a4 | | Colorless liquid | 4° C. |
| Insulin-10 | | Colorless liquid | 4° C. |

Among them, Degludec means insulin degludec, Icodec means A14E, B16H, B25H, B29K(N^{ε}-eicosandioyl-gGlu-2xOEG), DesB30 human insulin analog, Insulin-a1 means the long-acting insulin A14E, B16E, B25H, B29K (N^{ε}-eicosandioyl-gGlu-2xOEG), DesB30 human insulin analog disclosed in CN105636979A. Insulin-a3 means A14E, B16E, B25H, B29K(N(ε)-COOH(CH₂)₁₈CO-L-Lys-CO (CH₂)₂CO—(OEG)₂), desB30 human insulin analog. Insulin-a4 means A14E, B16E, B25H, B29K(N(ε)-COOH(CH₂)₁₆CO-L-Lys-CO (CH₂)₂CO—(OEG)₂), desB30 human insulin analog. Insulin-a10 means A14E, B16H, B25H, B29K(N(ε)-COOH(CH₂)₁₈CO-L-Lys-CO (CH₂)₂CO—(OEG)₂), desB30 human insulin analog.

### (2) Sample Configuration

The different insulin analog APIs used in the pharmacological experiments were formulated to the desired concentrations using PBS buffer solution.

### (3) Experimental Animals

TABLE 3

| Species | C57BL/6 mice |
|---|---|
| Level | SPF |
| Weight range of ordered | 20-24 g |
| Gender | Male |
| Supplier | Hunan SJA Laboratory Animal Co., |
| | Ltd |
| Supplier's address | Hunan, China |
| Method of animal identification | Mark the tail with a marker |
| Number of animals ordered | 40 |
| Number of animals used | 36 |

### (4) Experimental Method

SPF grade C57BL/6 mice were reared in a suitable rearing box in a barrier environment, with a rearing temperature of 20-26° C., a humidity of 40-70%, a time between day and night of 12 h/12 h, and the mice had free access to standard food and autoclaved sterilization water. After a 3-day quarantine period and a 2-day acclimation period, random blood glucose was measured and mice were weighed. Mice were divided into 6 groups according to random blood glucose and body weight. Animal grouping and administration are shown in Table 4:

TABLE 4

| | | | | Dosing | | |
|---|---|---|---|---|---|---|
| | | | Dosage | volume | Way of | Dosing |
| Group | Type | Number | (nmol/Kg) | (mL/Kg) | administration | frequency |
| Control | C57BL/6 | 6 | / | 10 | S.C. | Once |
| Degludec | C57BL/6 | 6 | 200 | 10 | S.C. | Once |
| Icodec | C57BL/6 | 6 | 1400 | 10 | S.C. | Once |
| Insulin-a1 | C57BL/6 | 6 | 1400 | 10 | S.C. | Once |
| Insulin-a3 | C57BL/6 | 6 | 1400 | 10 | S.C. | Once |
| Insulin-a4 | C57BL/6 | 6 | 1400 | 10 | S.C. | Once |
| Insulin-a10 | C57BL/6 | 6 | 1400 | 10 | S.C. | Once |

Single subcutaneous administration (S.C.) was used to administer the corresponding vehicle or drug. The control group was administered the vehicle PBS without fasting during the whole process, and the animals were allowed to eat and drink freely. Random blood glucose values of C57 mice were measured before administration and at 0.25, 0.5, 1, 2, 4, 6, 8, 10, 24, 48, 54, 72 and 96 hours after administration.

All raw data were entered into Excel files and expressed as Mean±SEM. Statistical analysis of data was performed using Graphpad Prism 7.0 software, one-way or two-way ANOVA comparison method, and P<0.05 was used as the criterion for significant differences.

### (5) Results

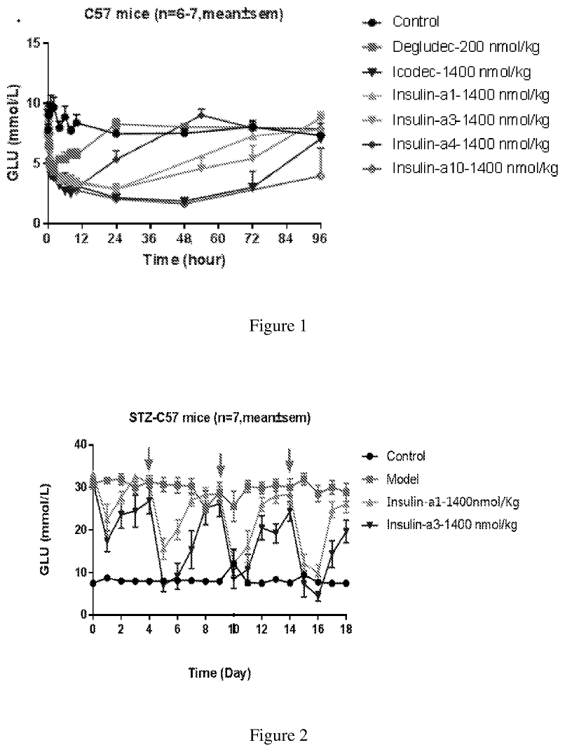

Compared with the control group, 1 h after administration, the blood glucose of the groups of insulin degludec, Icodec, Insulin-a1, Insulin-a3, Insulin-a4 and Insulin-a10 decreased significantly; 2 h after administration, the blood glucose of the mice in insulin degludec group reached the lowest level and then slowly increased, while the blood glucose of the mice in other 5 groups continued to decrease slowly; 10 h after administration, the blood glucose of the insulin degludec group had gradually recovered, and the blood glucose of the Insulin-a4 group had reached the lowest level and gradually recovered, the blood glucose of the groups of Icodec, Insulin-a1, Inslulin-a3 and Insulin-a10 still maintained a slow decline; 24 h after administration, the blood glucose of the mice in the insulin degludec group returned to normal, and the blood glucose of Insunlin-a4 group gradually recovered, the blood sugar of Insulin-a1 and Insulin-a3 groups reached the lowest level, and there was no significant difference between the two, and the blood glucose gradually recovered in the follow-up, while the blood glucose of Icodec and Insulin-a10 groups continued to decline slowly; 48 h after administration, the blood glucose of Insulin-a1 and Insulin-a4 groups returned to normal, the blood glucose of Insulin-a3 group showed an upward trend, but the blood glucose was still at a low level, the blood glucose of Icodec group reached the lowest level and gradually recovered, while the blood glucose of Insulin-a10 group continued to decrease slowly; 72 h after administration, the blood glucose of Insulin-a3 group remained low, the blood glucose of Icodec group gradually recovered, while the blood glucose of Insulin-a10 group reached the lowest level and then gradually increased; 96 h after administration, the blood glucose of other groups returned to normal level except for Insulin-a10 group, the blood glucose of Insulin-a10 group gradually increased, but it had not yet reached the normal level. The specific data are shown in Table 5 and FIG. 1.

TABLE 5
Effects of single administration on blood glucose of C57 mice (Mean ± SEM, n = 6)

| | Time point (h) | | | | | | |
|---|---|---|---|---|---|---|---|
| Groups | 0 | 0.25 | 0.5 | 1 | 2 | 4 | 6 |
| Control | 7.8 ± 1.1 | 9.0 ± 1.3 | 9.3 ± 1.9 | 9.8 ± 2.3 | 9.7 ± 2.1 | 8.0 ± 1.4 | 8.9 ± 2.2 |
| Degludec | 7.8 ± 0.8 | 6.5 ± 1.1* | 5.0 ± 0.4** | 4.7 ± 1.1** | 4.4 ± 1.3** | 5.4 ± 0.6** | 5.4 ± 1.2* |
| Insulin-a1 | 7.8 ± 1.1 | 8.8 ± 1.4 | 6.6 ± 0.7** | 5.6 ± 0.7** | 4.4 ± 0.7*** | 3.6 ± 0.7*** | 3.8 ± 0.4*** |
| Insulin-a3 | 8.0 ± 1.0 | 8.6 ± 1.0 | 6.2 ± 0.4** | 4.9 ± 0.8** | 4.5 ± 1.0*** | 3.9 ± 0.7*** | 3.7 ± 0.5*** |
| Insulin-a4 | 8.0 ± 1.3 | 9.0 ± 1.9 | 6.2 ± 1.6* | 5.0 ± 0.5** | 4.8 ± 0.5*** | 3.9 ± 0.5*** | 3.8 ± 1.1** |
| Insulin-a10 | 9.1 ± 1.5* | 7.9 ± 1.5* | | 4.4 ± 0.6*** | 4.4 ± 1.3*** | 3.7 ± 1.0*** | 3.5 ± 1.1*** |

| | Time point (h) | | | | | |
|---|---|---|---|---|---|---|
| Groups | 8 | 10 | 24 | 48 | 72 | 96 |
| Control | 7.7 ± 0.9 | 8.4 ± 1.4 | 7.5 ± 0.9 | 7.5 ± 0.9 | 8.0 ± 1.2 | 7.3 ± 0.6 |
| Degludec | 5.8 ± 0.8** | 5.7 ± 0.9* | 8.3 ± 0.9 | 8.0 ± 0.3 | 8.0 ± 0.8 | 7.9 ± 0.3 |
| Insulin-a1 | 3.6 ± 0.7*** | 3.4 ± 0.7*** | 2.9 ± 0.7*** | 4.9 ± 1.5** | 7.3 ± 1.3 | 7.8 ± 0.6 |
| Insulin-a3 | 3.8 ± 0.5*** | 3.5 ± 0.7*** | 2.9 ± 1.0*** | 4.2 ± 2.0** | 5.4 ± 2.6 | 8.8 ± 1.1 |
| Insulin-a4 | 3.2 ± 0.6*** | 3.0 ± 0.6*** | 5.3 ± 1.8* | 8.7 ± 0.9* | 8.0 ± 0.9 | 7.9 ± 1.1 |
| Insulin-a10 | 3.1 ± 1.0*** | 2.8 ± 0.7*** | 2.1 ± 0.7*** | 1.7 ± 0.6*** | 1.8 ± 0.1*** | 4.0 ± 4.0** |

Note:
*P < 0.05,
**P < 0.01,
***P < 0.001 vs Control

The results show that in this single administration experiment of normal C57BL/6 mice, the effective blood glucose control time of insulin degludec is 24 h, the effective blood glucose control time of Insulin-a4 is 48 h, and the effective blood glucose control time of Insulin-a1 is 72 h, the effective blood glucose control time of Icodec and Insulin-a3 are both 96 h, while the effective blood glucose control time of Insulin-a10 is more than 96 h. Compared with Icodec, although the effect of Insulin-a3 on blood glucose control is slightly worse, it still has the same effective blood glucose control time as Icodec, while the effect of Insulin-a10 on blood glucose control is consistent with the trend of Icodec and can be maintained for a longer time.

### Example 5: Hypoglycemic Effect of Test Drugs on STZ-Induced Type I Diabetes Mellitus (T1DM) of C57BL/6 Mice

### (1) Test Product

TABLE 6

| | | | Storage |
|---|---|---|---|
| Name | Supplier | Physical state | conditions |
| Insulin-a1 | Dongguan HEC | Colorless liquid | 4° C. |
| Insulin-a3 | Biopharmaceutical | Colorless liquid | 4° C. |
| | R&D Co., Ltd. | | |

### (2) Sample Configuration

The different insulin analog APIs used in the pharmacological experiments were formulated to the desired concentrations using PBS buffer solution.

### (3) Experimental Animals

TABLE 7

| Species | C57BL/6 mice |
|---|---|
| Level | SPF |
| Weight range of ordered | 20-24 g |
| Gender | Male |
| Supplier | Hunan SJA Laboratory Animal Co., Ltd |
| Supplier's address | Hunan, China |
| Method of animal identification | Mark the tail with a marker |
| Number of animals ordered | 40 |
| Number of animals used | 40 |

### (4) Experimental Method

SPF grade C57BL/6 mice were reared in a suitable rearing box in a barrier environment, with a rearing temperature of 20-26° C., a humidity of 40-70%, a time between day and night of 12 h/12 h, and the mice had free access to standard food and autoclaved sterilization water. After a 3-day quarantine period and a 2-day acclimation period, the mice were fasted for 12 h, and the mice were injected intraperitoneally with streptozotocin solution (STZ, 13 mg/mL, in citrate buffer) or citrate buffer at 130 mg/kg (control group). 3 Days and 7 days after administration of streptozotocin, random blood glucose and fasting blood glucose were detected, and the random blood glucose value above 25 mmol/L and fasting blood glucose value above 11.1 mmol/L were selected as T1DM model mice for follow-up experiments. On the day before administration, random blood glucose was monitored and mice were weighed. Mice were divided into 4 groups according to random blood glucose and body weight.

Animal grouping and administration are as follows:

TABLE 8

| | | | Dosage | Dosing | drug- | |
|---|---|---|---|---|---|---|
| | | num- | (nmol/ | volume | delivery | Dosing |
| Group | Type | ber | Kg) | (mL/Kg) | way | frequency |
| Control | C57BL/6 | 6 | / | 10 | S.C. | Four times |
| Model | C57BL/6 | 7 | / | 10 | S.C. | Four times |
| Insulin-a1 | C57BL/6 | 7 | 1400 | 10 | S.C. | Four times |
| Insulin-a3 | C57BL/6 | 7 | 1400 | 10 | S.C. | Four times |

Subcutaneous administration (S.C.) was used to administer the corresponding vehicle or drug, once every 4-5 days, for a total of 4 administrations. During the experiment, the animals were allowed to eat and drink water freely. The random blood glucose before the first administration, and 0.25, 0.5, 1, 2, 4, 6, 8, 10, 24, 48, 72, and 96 h after administration were assessed, as well as the random blood glucose before the second, third and fourth administration, and 1, 2, 4, 6, 8, 24, 48, 72, 96 and 120 h after administration.

All raw data were entered into Excel files and expressed as Mean±SEM. Statistical analysis of data was performed using Graphpad Prism 7.0 software, one-way or two-way ANOVA comparison method, and P<0.05 was used as the criterion for significant differences.

### (5) Results

The specific data are shown in Table 9 and FIG. 2.

TABLE 9

| | Time point (Day) | | | | | | | |
|---|---|---|---|---|---|---|---|---|
| | 0 | | 4 | | 9 | | 14 | |
| Group | (1st) | 1 | (2nd) | 5 | (3rd) | 10 | (4th) | 18 |
| Control | 7.5 ± 0.5 | 8.8 ± 0.5 | 8.0 ± 0.4 | 8.0 ± 0.7 | 7.9 ± 0.5 | 12.2 ± 8.3 | 7.6 ± 0.8 | 7.5 ± 0.6 |
| Model | 31.1 ± 3.8 | 31.7 ± 2.0 | 31.4 ± 3.7 | 30.7 ± 4.5 | 28.8 ± 6.2 | 29.0 ± 3.5 | 30.1 ± 5.2 | 28.9 ± 5.6 |
| Insulin-a1 | 33.3 ± 0.1 | 22.5 ± 9.6* | 31.1 ± 2.3 | 15.6 ± 6.6*** | 28.4 ± 4.9 | 12.2 ± 7.9*** | 28.4 ± 5.9 | 26.2 ± 5.7 |
| Insulin-a3 | 31.3 ± 3.1 | 17.4 ± 6.7*** | 26.9 ± 8.0 | 7.1 ± 3.8*** | 26.2 ± 7.9 | 8.6 ± 5.7*** | 24.4 ± 5.7 | 19.7 ± 6.4* |

Note:
*P < 0.05,
***P < 0.001 vs Model

The results showed that compared with the model group, the blood glucose of Insulin-a1 decreased significantly after 24 h of each administration, reaching the lowest level and then slowly increased, and reaching the normal level after 72 h of administration; 24 h after the first and second administrations, the blood glucose of Insulin-a3 decreased significantly, and reaching the lowest level, then slowly increased, and reaching the normal level after 96 h of administration. With the number of administrations increasing, the effective glucose control time of Insulin-a3 was prolonged after the third and fourth administrations, and reaching the normal level only after 120 h of administration, and after each administration, the lowering effect on blood glucose of Insulin-a3 was better than that of Insulin-a1.

In conclusion, the glucose control effect and effective glucose control time of Insulin-a3 were significantly better than those of Insulin-a1.

### Example 6: Hypoglycemic Effect of Test Drugs on STZ-Induced Type I Diabetes Mellitus (T1DM) of C57BL/6 Mice

### (1) Test Product

TABLE 10

| | | | Storage |
|---|---|---|---|
| Name | Supplier | Physical state | conditions |
| Icodec | Dongguan HEC | Colorless liquid | 4° C. |
| Insulin-a3 | Biopharmaceutical | Colorless liquid | 4° C. |
| Insulin-a10 | R&D Co., Ltd. | Colorless liquid | 4° C. |

### (2) Sample Configuration

The different insulin analog APIs used in the pharmacological experiments were formulated to the desired concentrations using PBS buffer solution.

### (3) Experimental Animals

TABLE 11

| Species | C57BL/6 mice |
|---|---|
| Level | SPF |
| Weight range of ordered | 20-24 g |
| Gender | Male |
| Supplier | Hunan SJA Laboratory Animal Co., Ltd |
| Supplier's address | Hunan, China |
| Method of animal identification | Mark the tail with a marker |
| Number of animals ordered | 350 |
| Number of animals used | 60 |

### (4) Experimental Method

SPF grade C57BL/6 mice were reared in a suitable rearing box in a barrier environment, with a rearing temperature of 20-26° C., a humidity of 40-70%, a time between day and night of 12 h/12 h, and the mice had free access to standard food and autoclaved sterilization water. After the 3-day quarantine period and the 2-day acclimation period, the mice were fasted for 12 h, and the mice were injected intraperitoneally with streptozotocin solution (STZ, 13 mg/mL, in citrate buffer) at 130 mg/kg. 3 Days and 7 days after administration of streptozotocin, random blood glucose and fasting blood glucose were detected, and the random blood glucose value above 25 mmol/L and fasting blood glucose value above 11.1 mmol/L were selected as T1DM model mice for follow-up experiments. On the day before administration, random blood glucose was monitored and mice were weighed. Mice were divided into 6 groups according to random blood glucose and body weight.

Animal grouping and administration are shown in Table 12:

TABLE 12

| | | | | Dosing | drug- | |
|---|---|---|---|---|---|---|
| | | | Dosage | volume | delivery | Dosing |
| Group | Type | number | (nmol/Kg) | (mL/Kg) | way | frequency |
| Model | C57BL/6 | 7 | / | 10 | S.C. | Three times |
| Icodec-250 | C57BL/6 | 7 | 250 | 10 | S.C. | Three times |
| Icodec-500 | C57BL/6 | 7 | 500 | 10 | S.C. | Three times |
| Icodec-1000 | C57BL/6 | 7 | 1000 | 10 | S.C. | Three times |
| HEC-Insulin-a3 | C57BL/6 | 7 | 500 | 10 | S.C. | Three times |
| HEC-Insulin-a10 | C57BL/6 | 7 | 500 | 10 | S.C. | Three times |

Subcutaneous administration (S.C.) was used to administer the corresponding vehicle or drug, once every 4-5 days, for a total of 3 administrations. During the experiment, the animals were allowed to eat and drink water freely. The random blood glucose before the first administration, and 0.25, 0.5, 1, 2, 4, 6, 8, 10, 24, 48, 72, and 96 h after administration were assessed, as well as the random blood glucose before the second and third administration, and 0.5, 1, 2, 6, 24, 48, 72, 96 and 120 h after administration.

All raw data were entered into Excel files and expressed as Mean±SEM. Statistical analysis of data was performed using Graphpad Prism 7.0 software, one-way or two-way ANOVA comparison method, and P<0.05 was used as the criterion for significant differences.

### (5) Results

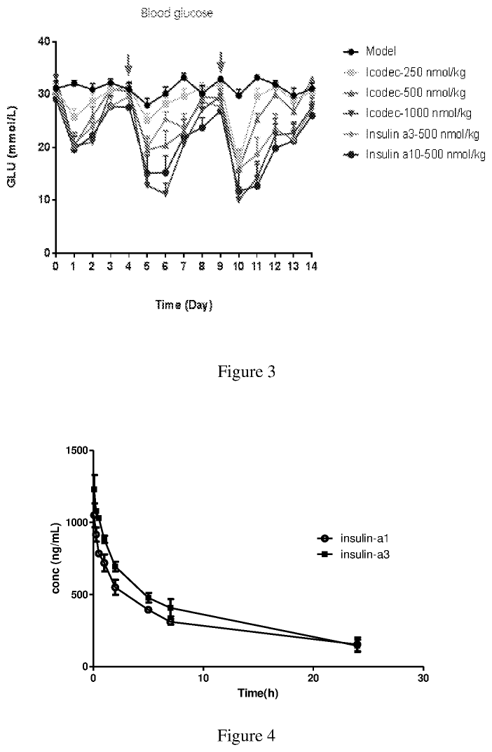

The specific data are shown in Table 13 and FIG. 3.

TABLE 13
Effects of repeated administration on random blood glucose in type I diabetic mice (Mean ± SEM, n = 7)

| | Time point (Day) | | | | | | |
|---|---|---|---|---|---|---|---|
| | 0 | | 4 | | 9 | | |
| Group | (1st) | 1 | (2nd) | 5 | (3rd) | 10 | 14 |
| Model | 32.0 ± 1.7 | 32.1 ± 1.5 | 31.0 ± 3.5 | 28.0 ± 3.5 | 33.0 ± 0.7 | 29.8 ± 3.1 | 31.1 ± 2.9 |
| Icodec-250 | 32.5 ± 2.2 | 25.7 ± 4.2 ** | 31.3 ± 2.6 | 25.1 ± 3.2 | 31.8 ± 2.2 | 17.2 ± 6.8 *** | 30.6 ± 4.6 |
| Icodec-500 | 32.5 ± 1.6 | 20.3 ± 6.3 *** | 30.6 ± 2.6 | 19.5 ± 5.8 ** | 27.0 ± 7.1* | 15.9 ± 6.0 *** | 33.0 ± 0.9 |
| Icodec-1000 | 30.8 ± 2.9 | 20.4 ± 4.6 *** | 31.6 ± 2.1 | 12.8 ± 5.4 *** | 29.7 ± 3.7* | 10.1 ± 6.4 *** | 27.7 ± 4.6 |
| Insulin-a3 | 31.8 ± 3.2 | 20.7 ± 5.2 *** | 29.7 ± 3.7 | 20.1 ± 5.4 ** | 27.7 ± 6.2* | 16.0 ± 9.4 ** | 27.4 ± 6.3 |
| Insulin-a10 | 32.0 ± 1.7 | 19.7 ± 6.6 *** | 27.6 ± 4.4 | 15.2 ± 8.9 ** | 26.6 ± 5.7* | 11.7 ± 9.4 *** | 26.1 ± 6.2# |

Note:
*P < 0.05,
** P < 0.01,
*** P < 0.001 vs Model.
Compared with the model group, the blood glucose of the three doses of Icodec-250, 500 and 1000 nmol/kg decreased significantly after 24 h of each administration, reaching the lowest level, and then slowly increased. During the whole experimental period, the lowering effect of Icodec on blood glucose and the effective blood glucose control time were in a dose-dependent manner, that is, the higher the dose, the stronger the lowering effect and the longer the time of blood glucose control. At the dose of 1000 nmol/kg, the effective blood glucose control time can reach 96 h.

Compared with the model group, the blood glucose of Insulin-a3 decreased significantly after 24 h of each administration, reaching the lowest level, and then slowly increased, the blood glucose returned to normal level 96 h after the first and second administrations. With the number of administrations increasing, the effective glucose control time of Insulin-a3 was prolonged after the third administration, and reached the normal level only after 120 h of administration. At the same time, the blood glucose of Insulin-a10 decreased significantly after 24 h of each administration, reaching the lowest level, then slowly increased, the blood glucose returned to normal level 96 h after the first administration, with the number of administrations increasing, the effective glucose control time of Insulin-a10 was prolonged after the second and third administrations, and reached normal level only after 120 h of administration. Compared with the Icodec-1000 nmol/kg group, the glucose control effect of Insulin-a3 was slightly worse, but better than that of the Icodec-500 nmol/kg group, and its effective glucose control time could be maintained for 96-120 h; the glucose control effect of Insulin-a10 was equivalent to that of Icodec-1000 nmol/kg, and its effective glucose control time can be maintained for 120 h.

In conclusion, Insulin-a3 and Insulin-a10 can still achieve equivalent or better hypoglycemic effect when the dose is lower than twice of Icodec.

### Example 7: PK Test of Intravenous Injection in Rats

### (1) Test Product

TABLE 14

| | | | Storage |
|---|---|---|---|
| Name | Supplier | Physical state | conditions |
| Insulin-a1 | Dongguan HEC | Colorless liquid | 4° C. |
| Insulin-a3 | Biopharmaceutical | Colorless liquid | 4° C. |
| | R&D Co., Ltd. | | |

### (2) Sample Configuration

The different insulin analog APIs used in the pharmacological experiments were formulated to the desired concentrations using PBS buffer solution.

### (3) Experimental Animals

TABLE 15

| Species | SD rat |
|---|---|
| Level | SPF |
| Weight range | 330-370 g |
| Gender | Male |
| Supplier | Hunan SJA Laboratory Animal Co., Ltd |
| Supplier's address | Hunan, China |
| Method of animal identification | Mark the base of the tail with a marker |
| Number of animals used | 4 |

### (4) Experimental Method

4 Male SD rats (2/group) were administered a single intravenous (i.v.) dose of 10 nmol/kg Insulin-a1 or Insulin-a3, blood was collected and plasma was centrifuged at 0.083, 0.25, 0.5, 1, 2, 5, 7, 24 h after administration, and the concentration of Insulin-a1 or Insulin-a3 in plasma was detected by LC-MS/MS method.

### (5) Experimental Results

The results in FIG. 4 and Table 16 showed that compared with Insulin-a1, the AUClast and Cmax of Insulin-a3 were slightly higher, and the higher Cmax indicated that the plasma binding may be higher. In addition, the half-lives of Insulin-a1 and Insulin-a3 were 15.3±4.8 h and 11.2±1.9 h, respectively. In conclusion, Insulin-a3 and Insulin-a1 have similar effects on PK in rats.

TABLE 16
In vivo I.V. PK data table of SD rats

| | Cₘₐₓ(ng/mL) | | AUCₗₐₛₜ(ng*h/mL) | | T_{1/2} (h) | |
|---|---|---|---|---|---|---|
| Test compound | Mean | SD | Mean | SD | Mean | SD |
| Insulin-a1 | 1050 | 83 | 7550 | 660 | 15.3 | 4.8 |
| Insulin-a3 | 1230 | 99 | 9170 | 1100 | 11.2 | 1.9 |

### Example 8: PK Test of In Vivo Subcutaneous Injection in Rats

### (1) Test Product

TABLE 17

| | | | Storage |
|---|---|---|---|
| Name | Supplier | Physical state | conditions |
| Icodec | Dongguan HEC | Colorless | 4° C. |
| | Biopharmaceutical | liquid | |
| Insulin-a1 | R&D Co., Ltd. | Colorless | 4° C. |
| | | liquid | |
| Insulin-a10 | | Colorless | 4° C. |
| | | liquid | |

### (2) Sample configuration

The different insulin analog APIs used in the pharmacological experiments were formulated to the desired concentrations using PBS buffer solution.

### (3) Experimental Animals

TABLE 18

| Species | SD rat |
|---|---|
| Level | SPF |
| Weight range | 220-340 g |
| Gender | Male |
| Supplier | Hunan SJA Laboratory Animal Co., Ltd |
| Supplier's address | Hunan, China |
| Method of animal identification | Mark the base of the tail with a marker |
| Number of animals used | 9 |

### (4) Experimental Method

9 SD rats (3/group) were administered a single subcutaneous (SC.) dose of 10 nmol/kg Insulin-a1, Insulin-a10 and Icodec, blood was collected and plasma was centrifuged at 1 h, 2 h, 5 h, 24 h, 31 h, 48 h, 72 h, 96 h and 120 h, the concentrations of Insulin-a1, Insulin-a3 and Icodec in plasma were detected.

### (5) Experimental Results

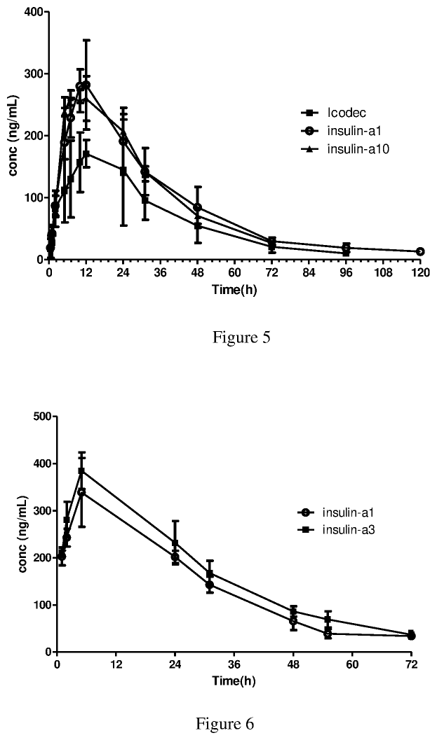

The results in FIG. 5 and Table 19 showed that compared with Icodec, the AUClast and Cmax of Insulin-a1 and Insulin-a10 were slightly higher, and the higher Cmax indicated that the plasma binding may be higher. In addition, the subcutaneous half-lives of Insulin-a1 and Insulin-a10 were 21 h and 17.2 h, respectively. In conclusion, the effect in PK of Insulin-a10 on mice is better than that of the control Icodec.

TABLE 19
Subcutaneous SC. PK data table of SD rats

| Test | Cₘₐₓ(ng/mL) | | AUCₗₐₛₜ(ng*h/mL) | | MRTINF_obs(h) | | T_{1/2} (h) | |
|---|---|---|---|---|---|---|---|---|
| compound | Mean | SD | Mean | SD | Mean | SD | Mean | SD |
| Icodec | 183 | 34 | 6560 | 2500 | 30.5 | 4.4 | 16.7 | 1.3 |
| Insulin-a1 | 313 | 46 | 10300 | 1900 | 34.1 | 4.6 | 21 | 2.8 |
| Insulin-a10 | 280 | 14 | 9350 | 150 | 29.2 | 1.2 | 17.2 | 1.0 |

### Example 9: PK Test of Subcutaneous Injection in C57BL6 Mice

### (1) Test Product

TABLE 20

| | | | Storage |
|---|---|---|---|
| Name | Supplier | Physical state | conditions |
| Insulin-a1 | Dongguan HEC | Colorless liquid | 4° C. |
| Insulin-a3 | Biopharmaceutical | Colorless liquid | 4° C. |
| | R&D Co., Ltd. | | |

### (2) Sample Configuration

The different insulin analog APIs used in the pharmacological experiments were formulated to the desired concentrations using PBS buffer solution.

### (3) Experimental Animals

TABLE 21

| Species | C57BL6 mice |
|---|---|
| Level | SPF |
| Weight range | 23-25 g |
| Gender | Male |
| Supplier | Hunan SJA Laboratory Animal Co., Ltd |
| Supplier's address | Hunan, China |
| Method of animal identification | Mark the base of the tail with a marker |
| Number of animals used | 6 |

### (4) Experimental Method

6 C57 mice (3/group) were administered a single subcutaneous (SC.) dose of 10 nmol/kg Insulin-a1 or Insulin-a3, blood was collected and plasma was centrifuged at 1 h, 2 h, 5 h, 24 h, 31 h, 55 h and 72 h after administration, the concentration of Insulin-a1 or Insulin-a3 in plasma was detected.

### (5) Experimental Results

The results in FIG. 6 and Table 22 showed that compared with Insulin-a1, the AUCₗₐₛₜ and Cₘₐₓ of Insulin-a3 were slightly higher, and the higher Cₘₐₓ indicated that the plasma binding may be higher, and in the insulin receptor activity test with 1.5% HAS added, it was also proved that Insulin-a3 had better albumin binding effect, reflecting a longer duration of efficacy. In addition, the subcutaneous half-lives of Insulin-a1 and Insulin-a3 were 14.3 h and 18.6 h, respectively. In conclusion, the effect in PK of Insulin-a3 in mouse is not inferior to that of the control Insulin-a1.

TABLE 22
Subcutaneous SC. PK data table of C57BL6 mice

| Test | Cₘₐₓ(ng/mL) | | AUCₗₐₛₜ(ng*h/mL) | | T_{1/2} (h) | |
|---|---|---|---|---|---|---|
| compound | Mean | SD | Mean | SD | Mean | SD |
| Insulin-a1 | 339 | 73 | 10000 | 1500 | 14.3 | 3.7 |
| Insulin-a3 | 385 | 39 | 12300 | 1600 | 18.6 | 3.1 |

### Example 10: PK Experiment of Beagle Dog

### (1) Test Product

TABLE 23

| | | | Storage |
|---|---|---|---|
| Name | Supplier | Physical state | conditions |
| Icodec | Dongguan HEC | Colorless liquid | 4° C. |
| Insulin-a10 | Biopharmaceutical | Colorless liquid | 4° C. |
| | R&D Co., Ltd. | | |

### (2) Sample Configuration

The different insulin analog APIs used in the pharmacological experiments were formulated to the desired concentrations using PBS buffer solution.

### (3) Experimental Animals

TABLE 24

| Species | Beagle dogs |
|---|---|
| Level | Ordinary grade |
| Weight range | 9~11 kg |
| Gender | Male |
| Supplier | Beijing Marshall Biotechnology Co., |
| | Ltd. |
| Supplier's address | Beijing, China |
| Method of animal identification | Ear number |
| Number of animals used | 2 |

### (4) Experimental Method

Two beagle dogs, one in each group, a double-cycle crossover design was used, with a washout period of 1 week, and a single dose of 10 nmol/kg of Icodec or Insulin-a10 was administered to the lateral small saphenous vein of the hind limb in each cycle, the blood was collected and plasma was centrifuged at 0.083, 0.25, 0.5, 1, 2, 6, 8, 24, 30, 48, 72 and 96 h after administration, the concentration of Icodec or Insulin-a10 in the plasma was detected.

### (5) Experimental Results

TABLE 25
I.V. PK data tabel of Beagle dogs

| Test compound | Cₘₐₓ (ng/ml) | AUCₗₐₛₜ (ng*h/mL) | T_{1/2} (h) |
|---|---|---|---|
| Icodec | 1240 ± 170 | 35700 ± 120 | 35.7 ± 6.7 |
| Insulin-a10 | 1170 ± 14 | 28300 ± 3500 | 46 ± 10.8 |

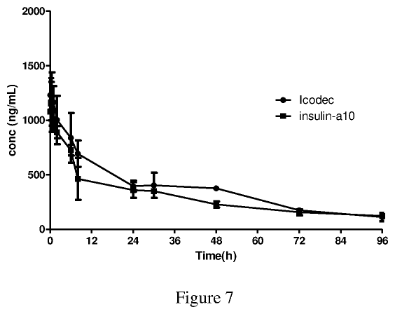

The results in Table 25 and FIG. 7 showed that compared with Icodec, the Cₘₐₓ of Insulin-a10 was comparable, and the AUCₗₐₛₜ was slightly lower, while the half-life of Insulin-a10 was 46±10.8 h, which was significantly higher than that of Icodec of 35.7±6.7 h. In conclusion, the Cₘₐₓ of Insulin-a10 in Beagles is comparable to that of Icodec, and the half-life is longer.

Reference throughout this specification to “an embodiment,” “some embodiments,” “one embodiment”, “another example,” “an example,” “a specific example,” or “some examples,” means that a particular feature, structure, material, or characteristic described in connection with the embodiment or example is included in at least one embodiment or example of the present disclosure. Thus, the appearances of the phrases such as “in some embodiments,” “in one embodiment”, “in an embodiment”, “in another example, “in an example,” “in a specific examples,” or “in some examples,” in various places throughout this specification are not necessarily referring to the same embodiment or example of the present disclosure. Furthermore, the particular features, structures, materials, or characteristics may be combined in any suitable manner in one or more embodiments or examples. In addition, those skilled in the art can integrate and combine different embodiments, examples or the features of them as long as they are not contradictory to one another.

Although explanatory embodiments have been shown and described, it would be appreciated by those skilled in the art that the above embodiments cannot be construed to limit the present disclosure, and changes, alternatives, and modifications can be made in the embodiments without departing from spirit, principles and scope of the present disclosure.

1. A novel side chain compound having the structure shown in formula (I):
W—X-Y-Z—R (I)
wherein:
W is a fatty acid or fatty diacid with 10-20 carbon atoms, the structure is —CO(CH₂)ₙCOOH, and n is an integer between 10-20;
X is a diamino compound containing a carboxylic acid group, wherein the carbon atom connecting the carboxylic acid group can be a chiral carbon or an achiral carbon, and has the structures shown in formulas (a1), (a2) and (a3), 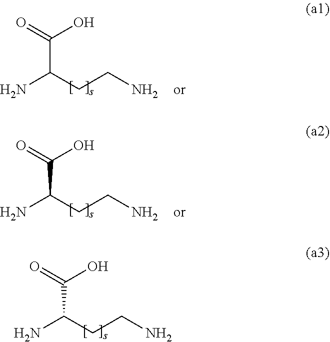
wherein s is an integer between 2-20, preferably 2-10, more preferably 2-8, and one of the amino groups in X is connected with one of the acyl groups in W to form an amide bond;
Y is -A(CH₂)ₘB—, wherein m is an integer between 1-10, preferably an integer between 1-6, A and B are absent or are —CO—;
Z is —(OEG)ₚ, p is an integer between 1-3, preferably 2, and the OEG structure is 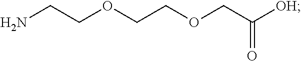 [SMILES: NCCOCCOCC(=O)O]
R is a leaving group, preferably an activated ester group;
the linking groups between W, X, Y and Z are amide peptide bonds or peptide bonds.

2. A novel side chain compound having the structure shown in formula (I):
W—X—Y—Z—R (I)
wherein:
W is a fatty acid or fatty diacid with 10-20 carbon atoms, the structure is —CO(CH₂)ₙCOOH, and n is an integer between 10-20;
X is a diamino compound containing a carboxylic acid group, wherein the carbon atom connecting the carboxylic acid group can be a chiral carbon or an achiral carbon, and has the structures shown in formulas (a1), (a2) and (a3), 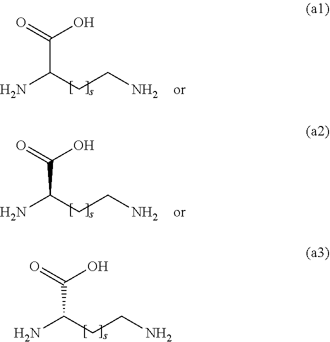
wherein s is an integer between 2-20, preferably 2-10, more preferably 2-8, and one of the amino groups in X is connected with one of the acyl groups in W to form an amide bond;
Y is -A(CH₂)ₘB—, wherein m is an integer between 1-10, preferably an integer between 1-6, A and B are absent or are —CO—;
Z is —(OEG)ₚ, p is an integer between 4-30, and the OEG structure is 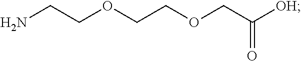 [SMILES: NCCOCCOCC(=O)O]
R is a leaving group, preferably an activated ester group;
the linking groups between W, X, Y and Z are amide peptide bonds or peptide bonds.

3. The compound of claim 1 having the following structural formulas: 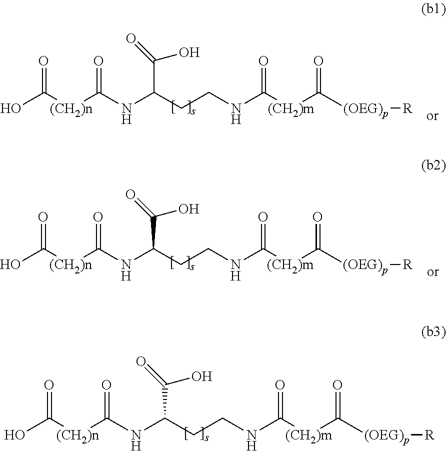
wherein, n is an integer between 14-20, s is an integer between 2-4, m is an integer between 1-4, p is 2,
R is selected from the following groups: 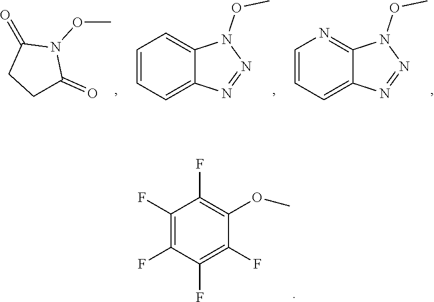
preferably, n is an integer between 16-18, s is an integer between 2-4, m is 2, p is 2,
preferably, R is:

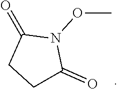

4. The compound of claim 1 selecting from any one of the following compounds: 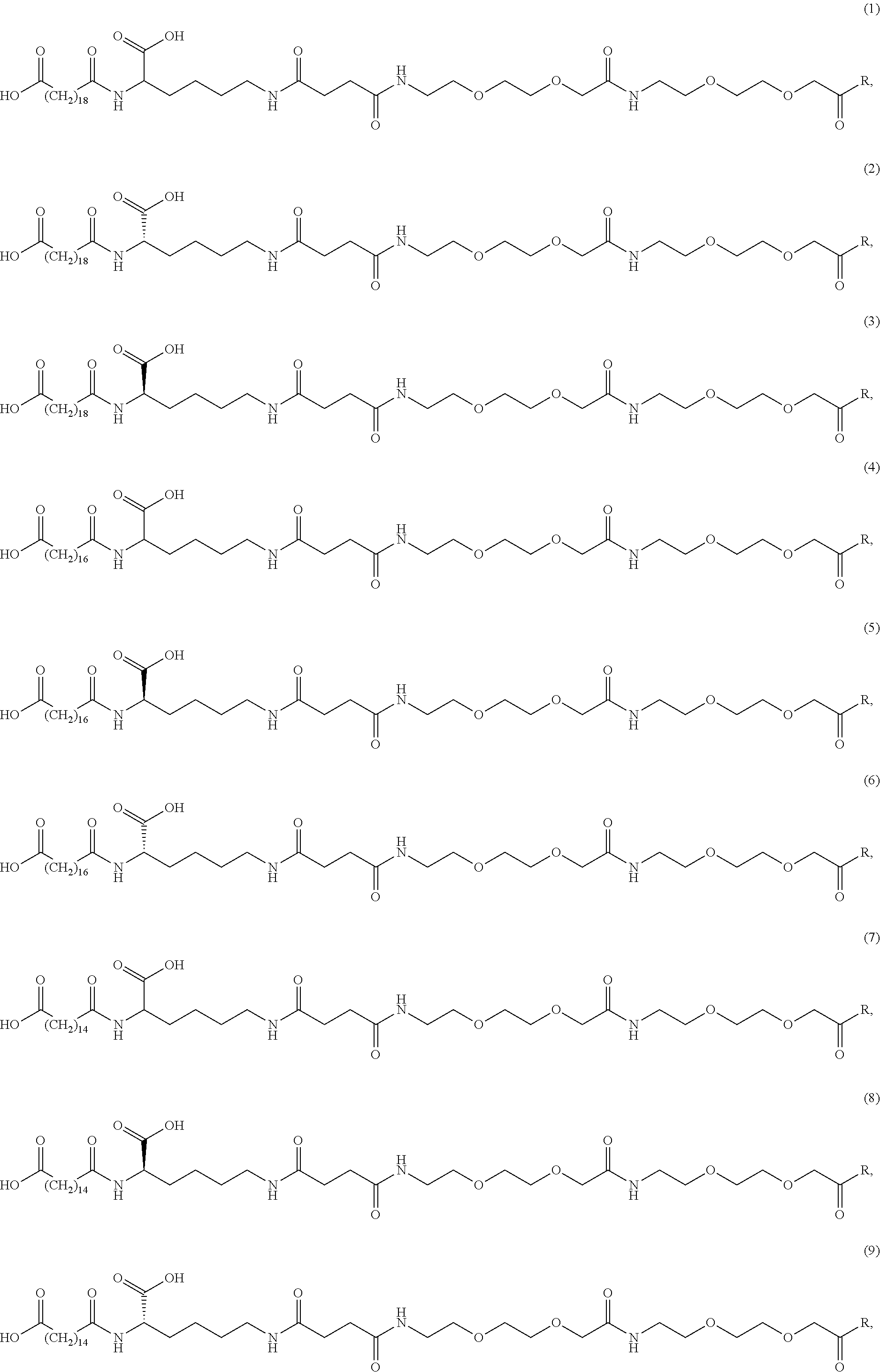 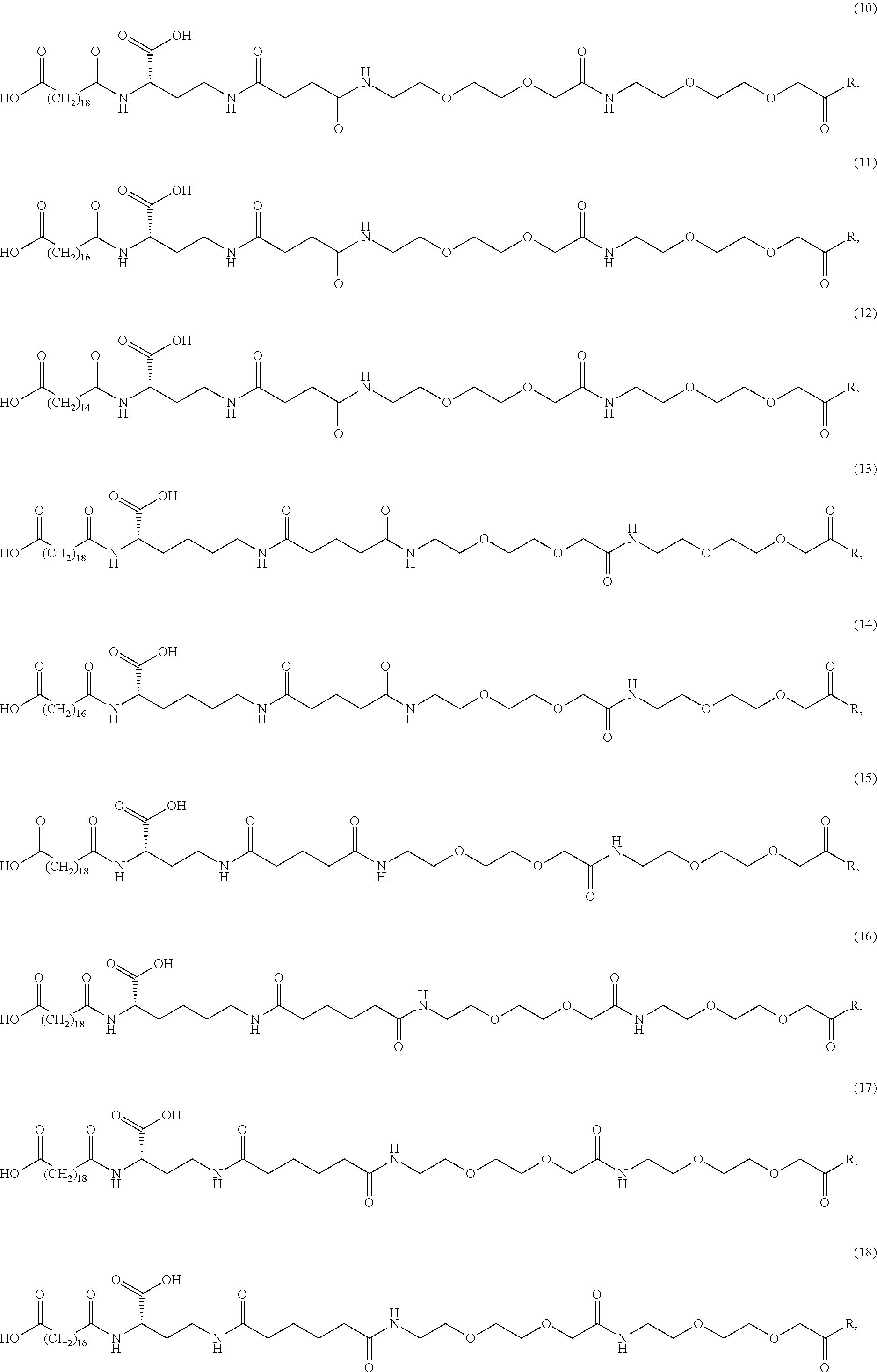
wherein, R is 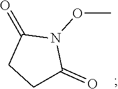
preferably, the compound has the following structural formulas: 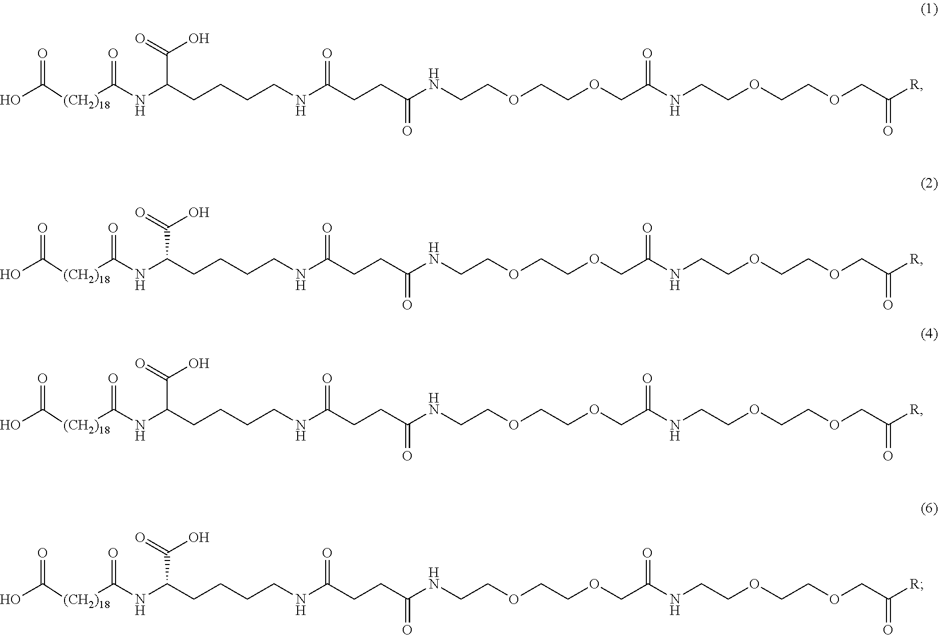
more preferably, the compound has the following structural formulas:

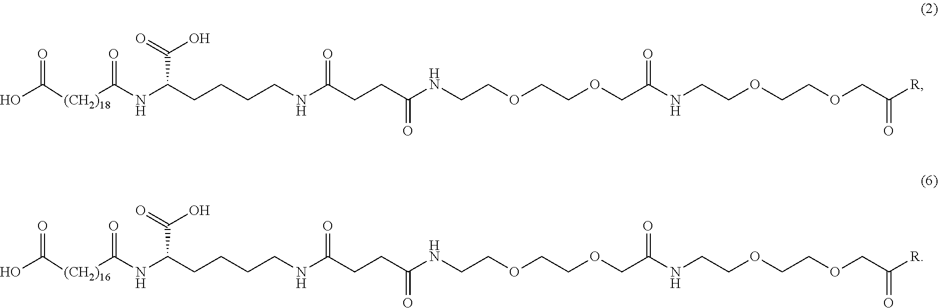

5. A novel acylated insulin analog obtained by an acylation reaction between the side chain compound of claim 1 and a human insulin analog, and having the structure shown in formula (II):
W—X—Y—Z-M (II)
wherein:
W is a fatty acid or fatty diacid with 10-20 carbon atoms, the structure is —CO(CH₂)ₙCOOH, and n is an integer between 10-20;
X is a diamino compound containing a carboxylic acid group, wherein the carbon atom connecting the carboxylic acid group can be a chiral carbon or an achiral carbon, and has the structures shown in formulas (a1), (a2) and (a3), 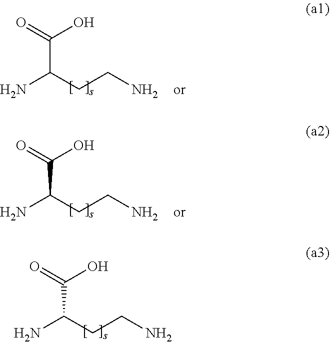
wherein s is an integer between 2-20, preferably 2-10, more preferably 2-8, and one of the amino groups in X is connected with one of the acyl groups in W to form an amide bond;
Y is -A(CH₂)ₘB—, wherein m is an integer between 1-10, preferably an integer between 1-6, A and B are absent or are —CO—;
Z is —(OEG)ₚ, p is an integer between 1-3, preferably 2, and the OEG structure is 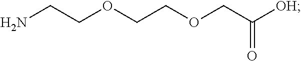 [SMILES: NCCOCCOCC(=O)O]
the linking groups between W, X, Y and Z are amide bonds or peptide bonds;
M is a human insulin analog.

6. The acylated insulin analog of claim 5, wherein the side chain compound has the following structures: 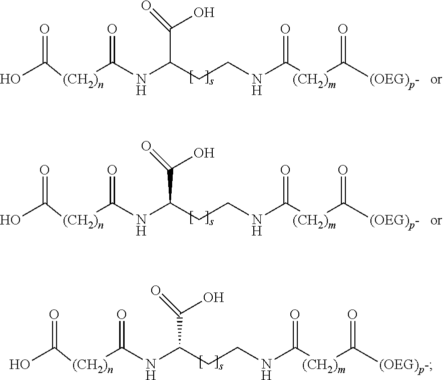
preferably, the side chain compound has the following structure: 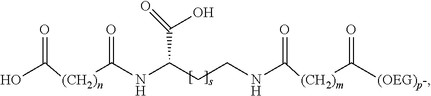
wherein, n is an integer between 14-20, s is an integer between 2-8, m is an integer between 1-6, and p is an integer between 1-3.

7. The acylated insulin analog of claim 5, wherein the human insulin analog M has A chain and B chain, the amino acid sequence of the A chain is shown in SEQ ID NO.1, the amino acid sequence of the B chain is shown in SEQ ID NO.2 or SEQ ID NO.3, and the human insulin analog is connected to the side chain compound by an amide bond through the F nitrogen of the lysine residue at position B29.

8. The acylated insulin analog of claim 5 having the following structural formulas:
A14E, B16E, B25H, B29K(N(ε)-COOH(CH₂)ₙCO—NHC(COOH)(CH₂)_{S}CH₂NH—CO(CH₂)ₘCO—(OEG)ₚ), desB30 human insulin analog, or,
A14E, B16H, B25H, B29K(N(ε)-COOH(CH₂)ₙCO—NHC(COOH)(CH₂)_{S}CH₂NH—CO(CH₂)ₘCO—(OEG)ₚ), desB30 human insulin analog;
wherein, n is an integer between 14-20, s is an integer between 2-8, m is an integer between 1-6, and p is 2.

9. The acylated insulin analog of claim 5 having the following structural formulas:
A14E, B16E, B25H, B29K(N(ε)-COOH(CH₂)ₙCO—NHC(COOH)(CH₂)_{S}CH₂NH—CO(CH₂)ₘCO—(OEG)ₚ), desB30 human insulin analog, or,
A14E, B16H, B25H, B29K(N(ε)-COOH(CH₂)ₙCO—NHC(COOH)(CH₂)_{S}CH₂NH—CO(CH₂)ₘCO—(OEG)ₚ), desB30 human insulin analog;
wherein, n is an integer between 14-18, s is an integer between 3-4, m is an integer between 2-4, and p is 2.

10. The acylated insulin analog of claim 6 selecting from any one of the following compounds:
A14E, B16E, B25H, B29K(N(ε)-COOH(CH₂)₁₈CO-Lys-CO(CH₂)₂CO—(OEG)₂), desB30 human insulin analog,
A14E, B16E, B25H, B29K(N(ε)-COOH(CH₂)₁₈CO-L-Lys-CO(CH₂)₂CO—(OEG)₂), desB30 human insulin analog,
A14E, B16E, B25H, B29K(N(ε)-COOH(CH₂)₁₈CO-D-Lys-CO(CH₂)₂CO—(OEG)₂), desB30 human insulin analog,
A14E, B16E, B25H, B29K(N(ε)-COOH(CH₂)₁₆CO-Lys-CO(CH₂)₂CO—(OEG)₂), desB30 human insulin analog,
A14E, B16E, B25H, B29K(N(ε)-COOH(CH₂)₁₆CO-D-Lys-CO(CH₂)₂CO—(OEG)₂), desB30 human insulin analog,
A14E, B16E, B25H, B29K(N(ε)-COOH(CH₂)₁₆CO-L-Lys-CO(CH₂)₂CO—(OEG)₂), desB30 human insulin analog,
A14E, B16E, B25H, B29K(N(ε)-COOH(CH₂)₁₄CO-Lys-CO(CH₂)₂CO—(OEG)₂), desB30 human insulin analog,
A14E, B16E, B25H, B29K(N(ε)-COOH(CH₂)₁₄CO-D-Lys-CO(CH₂)₂CO—(OEG)₂), desB30 human insulin analog,
A14E, B16E, B25H, B29K(N(ε)-COOH(CH₂)₁₄CO-L-Lys-CO(CH₂)₂CO—(OEG)₂), desB30 human insulin analog,
A14E, B16E, B25H, B29K(N(ε)-COOH(CH₂)₁₈CO-L-Dab-CO(CH₂)₂CO—(OEG)₂), desB30 human insulin analog,
A14E, B16E, B25H, B29K(N(ε)-COOH(CH₂)₁₆CO-L-Dab-CO(CH₂)₂CO—(OEG)₂), desB30 human insulin analog,
A14E, B16E, B25H, B29K(N(ε)-COOH(CH₂)₁₄CO-L-Dab-CO(CH₂)₂CO—(OEG)₂), desB30 human insulin analog,
A14E, B16E, B25H, B29K(N(ε)-COOH(CH₂)₁₈CO-L-Lys-CO(CH₂)₃CO—(OEG)₂), desB30 human insulin analog,
A14E, B16E, B25H, B29K(N(ε)-COOH(CH₂)₁₆CO-L-Lys-CO(CH₂)₃CO—(OEG)₂), desB30 human insulin analog,
A14E, B16E, B25H, B29K(N(ε)-COOH(CH₂)₁₈CO-L-Dab-CO(CH₂)₃CO—(OEG)₂), desB30 human insulin analog,
A14E, B16E, B25H, B29K(N(ε)-COOH(CH₂)₁₈CO-L-Lys-CO(CH₂)₄CO—(OEG)₂), desB30 human insulin analog,
A14E, B16E, B25H, B29K(N(ε)-COOH(CH₂)₁₈CO-L-Dab-CO(CH₂)₄CO—(OEG)₂), desB30 human insulin analog,
A14E, B16E, B25H, B29K(N(ε)-COOH(CH₂)₁₆CO-L-Dab-CO(CH₂)₄CO—(OEG)₂), desB30 human insulin analog,
A14E, B16H, B25H, B29K(N(ε)-COOH(CH₂)₁₈CO-Lys-CO(CH₂)₂CO—(OEG)₂), desB30 human insulin analog,
A14E, B16H, B25H, B29K(N(ε)-COOH(CH₂)₁₈CO-L-Lys-CO(CH₂)₂CO—(OEG)₂), desB30 human insulin analog,
A14E, B16H, B25H, B29K(N(ε)-COOH(CH₂)₁₈CO-D-Lys-CO(CH₂)₂CO—(OEG)₂), desB30 human insulin analog,
A14E, B16H, B25H, B29K(N(ε)-COOH(CH₂)₁₆CO-Lys-CO(CH₂)₂CO—(OEG)₂), desB30 human insulin analog,
A14E, B16H, B25H, B29K(N(ε)-COOH(CH₂)₁₆CO-D-Lys-CO(CH₂)₂CO—(OEG)₂), desB30 human insulin analog,
A14E, B16H, B25H, B29K(N(ε)-COOH(CH₂)₁₆CO-L-Lys-CO(CH₂)₂CO—(OEG)₂), desB30 human insulin analog,
A14E, B16H, B25H, B29K(N(ε)-COOH(CH₂)₁₄CO-Lys-CO(CH₂)₂CO—(OEG)₂), desB30 human insulin analog,
A14E, B16H, B25H, B29K(N(ε)-COOH(CH₂)₁₄CO-D-Lys-CO(CH₂)₂CO—(OEG)₂), desB30 human insulin analog,
A14E, B16H, B25H, B29K(N(ε)-COOH(CH₂)₁₄CO-L-Lys-CO(CH₂)₂CO—(OEG)₂), desB30 human insulin analog,
A14E, B16H, B25H, B29K(N(ε)-COOH(CH₂)₁₈CO-L-Dab-CO(CH₂)₂CO—(OEG)₂), desB30 human insulin analog,
A14E, B16H, B25H, B29K(N(ε)-COOH(CH₂)₁₆CO-L-Dab-CO(CH₂)₂CO—(OEG)₂), desB30 human insulin analog,
A14E, B16E, B25H, B29K(N(ε)-COOH(CH₂)₁₄CO-L-Dab-CO(CH₂)₂CO—(OEG)₂), desB30 human insulin analog,
A14E, B16H, B25H, B29K(N(ε)-COOH(CH₂)₁₈CO-L-Lys-CO (CH₂)₃CO—(OEG)₂), desB30 human insulin analog,
A14E, B16H, B25H, B29K(N(ε)-COOH(CH₂)₁₆CO-L-Lys-CO (CH₂)₃CO—(OEG)₂), desB30 human insulin analog,
A14E, B16H, B25H, B29K(N(ε)-COOH(CH₂)₁₈CO-L-Dab-CO(CH₂)₃CO—(OEG)₂), desB30 human insulin analog,
A14E, B16H, B25H, B29K(N(ε)-COOH(CH₂)₁₈CO-L-Lys-CO (CH₂)₄CO—(OEG)₂), desB30 human insulin analog,
A14E, B16H, B25H, B29K(N(ε)-COOH(CH₂)₁₈CO-L-Dab-CO(CH₂)₄CO—(OEG)₂), desB30 human insulin analog,
A14E, B16H, B25H, B29K(N(ε)-COOH(CH₂)₁₆CO-L-Dab-CO(CH₂)₄CO—(OEG)₂), desB30 human insulin analog;
preferably, the acylated insulin analog is selected from any one of the following compounds:
A14E, B16E, B25H, B29K(N(ε)-COOH(CH₂)₁₈CO-Lys-CO(CH₂)₂CO—(OEG)₂), desB30 human insulin analog,
A14E, B16E, B25H, B29K(N(ε)-COOH(CH₂)₁₈CO-L-Lys-CO(CH₂)₂CO—(OEG)₂), desB30 human insulin analog,
A14E, B16H, B25H, B29K(N(ε)-COOH(CH₂)₁₈CO-Lys-CO (CH₂)₂CO—(OEG)₂), desB30 human insulin analog;
A14E, B16H, B25H, B29K(N(ε)-COOH(CH₂)₁₈CO-L-Lys-CO (CH₂)₂CO—(OEG)₂), desB30 human insulin analog;
more preferably, the acylated insulin analog is selected from any one of the following compounds:
A14E, B16E, B25H, B29K(N(ε)-COOH(CH₂)₁₈CO-L-Lys-CO(CH₂)₂CO—(OEG)₂), desB30 human insulin analog,
A14E, B16H, B25H, B29K(N(ε)-COOH(CH₂)₁₈CO-L-Lys-CO (CH₂)₂CO—(OEG)₂), desB30 human insulin analog.

11. A pharmaceutical composition comprising the acylated insulin analog of claim 5.

12. (canceled)

13. (canceled)

14. A method of treating or preventing diabetes in a subject comprising administering to the subject a therapeutically effective amount of the pharmaceutical composition of claim 11;
wherein, the diabetes refers to type I and type II diabetes.

15. The method of claim 14, wherein the pharmaceutical composition is administered twice a week, once a week, or less frequently.

16. (canceled)

17. (canceled)

18. A method for preparing a novel acylated insulin analog of formula (II) in claim 5 comprising using the side chain compound of formula (I) and human insulin analog to carry out an acylation reaction;
wherein, the human insulin analog has A chain and B chain, the amino acid sequence of the A chain is shown in SEQ ID NO.1, the amino acid sequence of the B chain is shown in SEQ ID NO.2 or SEQ ID NO.3.
